(19) United States
(12) Patent Application Publication
(10) Pub. No.: US 2026/0363791 A1
(43) Pub. Date: Nov. 19, 2026

(54) SYSTEMS AND METHODS FOR REGISTERING INTRAVASCULAR AND EXTRAVASCULAR DATA

(71) Applicant: KONINKLIJKE PHILIPS N.V., EINDHOVEN (NL)

(72) Inventors: Eman NAMATI, CONCORD, MA (US); Damon DEPAOLI, ARLINGTON, MA (US); Jason TUCKER-SCHWARTZ, MELROSE, MA (US); Vivek WALIMBE, WESTFORD, MA (US); David VADER, CONCORD, MA (US)

(21) Appl. No.: 19/752,008

(22) Filed: Jul. 23, 2026

Related U.S. Application Data
(63) Continuation of application No. 19/118,687, filed on Oct. 6, 2023.
(60) Provisional application No. 63/414,360, filed on Oct. 7, 2022.
(60) Provisional application No. 63/540,847, filed on Sep. 27, 2023.

(51) Int. Cl. A61B 34/20; A61B 5/00; A61B 5/0215; A61B 5/026; G06T 5/60; G06T 5/70; G06T 7/00; G06T 7/33; G06T 7/60; G06T 7/73; G06T 11/00; A61B 90/00

(52) U.S. Cl. CPC A61B 34/20; A61B 5/0215; A61B 5/026; A61B 5/7207; A61B 5/743; G06T 5/60; G06T 5/70; G06T 7/0014; G06T 7/337; G06T 7/60; G06T 7/74; G06T 11/00; A61B 2034/2055; A61B 2034/2065; A61B 2090/372; G06T 2207/10048; G06T 2207/10101; G06T 2207/20081; G06T 2207/20084; G06T 2207/20092; G06T 2207/30021; G06T 2207/30048; G06T 2207/30101; G06T 2207/30204; G06T 2210/41

(57) ABSTRACT

Provided herein are systems and methods for registering extravascular and intravascular data.

### CROSS-REFERENCE

This application is a continuation of U.S. Patent Application No. 19/118,687, filed on Apr. 4, 2025, which is the U.S. national stage entry of International Patent Application No. PCT/US2023/076232, filed Oct. 6, 2023, which claims the benefit of U.S. Provisional Patent Application No. 63/414,360 filed on Oct. 7, 2022, and U.S. Provisional Patent Application No. 63/540,847 filed on Sep. 27, 2023, which are incorporated herein by reference in their entirety.

### BACKGROUND

Various modalities are utilized to image blood vessels of an individual, e.g., angiography (extravascular imaging) or other minimally invasive intravascular imaging modalities (e.g., intravascular ultrasound, optical coherence tomography). Each imaging modality provides a unique perspective of blood vessels when compared to the other, however there have been minimal advances in the real-time integration and/or co-registration of datatypes from the various modalities to improve vascular clinical procedures.

### SUMMARY

Described herein are methods and systems that register intravascular data to extravascular data, bridging the gap between intravascular and extravascular imaging modalities. In some embodiments, extravascular imaging comprises angiography. In some embodiments, intravascular imaging comprises optical coherence tomography, ultrasound, photo-acoustic tomography, spectroscopy, fluorescence, or any combination thereof.

Aspects of the disclosure provided herein comprise a method for displaying an object, comprising: acquiring intravascular data and extravascular data; determining a feature within the intravascular data and a feature within the extravascular data; registering the feature within the intravascular data and the feature within the extravascular data; and displaying an object relative to the registered feature within the intravascular data or the registered feature within the extravascular data, where the object is superimposed on a display of real-time extravascular data. In some embodiments, the feature within the intravascular data or the feature within the extravascular data are manually selected. In some embodiments, the feature within the intravascular data comprises a location within the intravascular data, and where the feature within the extravascular data comprises a location within the extravascular data. In some embodiments, the location within the intravascular data and the location within the extravascular data are automatically selected. In some embodiments, the object comprises a first object and a second object, where the first object and the second object are displayed relative to the registered location within the intravascular data or the registered location within the extravascular data. In some embodiments, the method comprises determining a location to guide the positioning of a foreign object. In some embodiments, the intravascular data comprises at least one image. In some embodiments, the extravascular data comprises at least one image. In some embodiments, the location within the intravascular data, the location within the extravascular location, the location to guide the positioning of the foreign object, or any combination thereof locations are determined by a predictive model. In some embodiments, the predictive model comprises a machine learning model. In some embodiments, the machine learning model comprises a neural network algorithm. In some embodiments, the method comprises guiding a catheter through a coronary artery to the object to treat coronary artery disease. In some embodiments, the catheter comprises an atherectomy catheter. In some embodiments, the method comprises guiding a catheter through a coronary artery to the object to diagnose coronary artery disease. In some embodiments, the catheter comprises a catheter to measure fractional flow reserve of the coronary artery. In some embodiments, the intravascular data comprises optical coherence tomography (OCT), intravascular ultrasound (IVUS), photoacoustic (PA), near infrared spectroscopy (NIRS), fluorescence, autofluorescence (AF), or any combination thereof data. In some embodiments, the intravascular data is detected by a multi-modal imaging system. In some embodiments, the multi-modal imaging system comprises a combined OCT and NIRS imaging system. In some embodiments, the intravascular data is detected by a one-dimensional sensing system. In some embodiments, the one-dimensional sensing system comprises a pressure sensing system. In some embodiments, the intravascular data comprises a measure of flow. In some embodiments, the real-time extravascular data is streamed directly from an x-ray system without transfer over a network to a processing unit configured to display the object superimposed on the display of the real-time extravascular data. In some embodiments, the location within the intravascular data, the location within the extravascular data, or the location to guide position of the foreign object comprise a location of: a blood vessel, any representation of blood vessel network, a side-branch of a blood vessel, a region to deploy a stent, a coronary plaque, a guidewire, a guide catheter, a stent, a distal or proximal location of an intravascular imaging pullback, a balloon, a valve, a clip, an atherectomy device, an intravascular data device, or any combination thereof. In some embodiments, the extravascular data comprises x-ray, CT, magnetic resonance, ultrasound, fluoroscopy, or any combination thereof image data. In some embodiments, the method comprises measuring heart cycle data from an external ECG signal, intravascular data, extravascular data, or any combination thereof, where the heart cycle data is used to improve an accuracy of the registration of the location within the intravascular data and the location within the extravascular data to the real-time extravascular data by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 96%, at least about 97%, at least about 98%, or at least about 99% compared to not measuring heart cycle data. In some embodiments, the location within the extravascular data is derived from an a priori selection, annotations, or a combination thereof from prior patient records. In some embodiments, the object comprises a fiducial marker, and where the spatial position of the fiducial marker is adjusted to account for motion artifact as the real-time extravascular data are displayed. In some embodiments, the method comprises measuring a distance from a catheter to the object. In some embodiments, the measured distance from the catheter to the object is displayed in real-time with a visual representation. In some embodiments, a fiducial location in the extravascular data is a feature that is not shown in the intravascular data. In some embodiments, the fiducial location comprises a radiopaque marker of a catheter. In some embodiments, the fiducial location comprises a known correlation to the intravascular data. In some embodiments, the known correlation comprises a distance. In some embodiments, the first object or the second object are displayed superimposed on the real-time extravascular data in one or more data views. In some embodiments, a first view of the one or more data views comprises a display of the real-time extravascular data without the display of the first object or the second object, and where a second view of the one or more data views comprises a display of the real-time extravascular data with the display of the first object or the second object. In some embodiments, a view of the one or more data views comprises a zoom view. In some embodiments, displaying the first object or the second object relative to the registered location within the intravascular data or the registered location within the extravascular data comprises a first state where the display of the first object or the second object is visible, or a second state where the display of the first object or the second object is not visible. In some embodiments, the display of the first object or the second object superimposed on the real-time extravascular data is displayed on one or more monitors. In some embodiments, the one or more monitors comprise an internal monitor positioned to face an operator of medical equipment, an external monitor positioned to face medical personnel using the medical equipment, or a combination thereof monitor configurations. In some embodiments, the internal monitor and the external monitor comprise different view configurations. In some embodiments, the one or more monitors comprise at least 2 external monitors positioned to face medical personnel using the medical equipment, where the at least 2 external monitors comprise different view configurations. In some embodiments, the method comprises displaying an indicator, where the indicator comprises a metric representing a distance between the object and a target location within the real-time extravascular data. In some embodiments, the target location is determined by at least one intravascular image or at least one extravascular image. In some embodiments, the method comprises processing a vessel geometry of the extravascular data and displaying the processed vessel geometry in a view of the one or more data views. In some embodiments, the extravascular data or the intravascular data is acquired without the use of contrast or with variable use of contrast.

Another aspect of the disclosure provided herein comprises a method, comprising: displaying an object relative to a feature of an intravascular dataset or a feature of an extravascular data, where the feature of the intravascular dataset and the feature of the extravascular data are registered to a real-time extravascular data, and where the object is superimposed on a display of the real-time extravascular data. In some embodiments, the feature of the intravascular data comprises a location within the intravascular dataset, and where the feature of the extravascular data comprises a location within the extravascular data. In some embodiments, the location within the intravascular data and the location within the extravascular data are automatically selected. In some embodiments, the object comprises a first object and a second object, where the first object and the second object are displayed relative to the registered location within the intravascular data or the registered location within the extravascular data. In some embodiments, the method comprises determining a location between the first object and the second object to guide placement of a stent. In some embodiments, the intravascular data comprises at least one image. In some embodiments, the extravascular data comprises at least one image. In some embodiments, the location within the intravascular data, the location within the extravascular data, the location between the first object and the second object, or any combination thereof locations is determined by a predictive model. In some embodiments, the predictive model comprises a machine learning model. In some embodiments, the machine learning model comprises a neural network algorithm. In some embodiments, the method comprises guiding a catheter through a coronary artery to the object to treat coronary artery disease. In some embodiments, the catheter comprises an atherectomy catheter. In some embodiments, the method comprises guiding a catheter through a coronary artery to the object to diagnose coronary artery disease. In some embodiments, the catheter comprises a catheter to measure fractional flow reserve of the coronary artery. In some embodiments, the intravascular data comprises optical coherence tomography (OCT), intravascular ultrasound (IVUS), photoacoustic (PA), near infrared spectroscopy (NIRS), or any combination thereof data. In some embodiments, the intravascular data is detected by a multi-modal imaging system. In some embodiments, the multi-modal imaging system comprises a combined OCT and NIRS imaging system. In some embodiments, the intravascular data is detected by a one-dimensional sensing system. In some embodiments, the one-dimensional sensing system comprises a pressure sensing system. In some embodiments, the intravascular data comprises a measure of flow. In some embodiments, the real-time extravascular data is streamed directly from an x-ray system without transfer over a network to a processing unit configured to display the object superimposed on the display of the real-time extravascular data. In some embodiments, the location within the intravascular data, the location within the extravascular data, or the location between the first object and the second object comprise a location of: a side-branch of a blood vessel, a region to deploy a stent, a coronary plaque, a guidewire, a guide catheter, a stent, a distal or proximal location of an intravascular imaging pullback, a balloon, a valve, a clip, an atherectomy device, an intravascular data device, or any combination thereof. In some embodiments, the extravascular data comprises x-ray, CT, magnetic resonance, ultrasound, fluoroscopy, or any combination thereof image data. In some embodiments, the method comprises measuring heart cycle data from an external ECG signal, intravascular data, extravascular data, or any combination thereof, where the heart cycle data is used to improve an accuracy of the registration of the location within the intravascular data and the location within the extravascular data to the real-time extravascular data by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 96%, at least about 97%, at least about 98%, or at least about 99% compared to not measuring heart cycle data. In some embodiments, the location within the extravascular data is derived from an a priori selection, annotations, or any combination thereof prior patient records. In some embodiments, the object comprises a fiducial marker, and where the spatial position of the fiducial marker is adjusted to account for motion artifact as the real-time extravascular data are displayed. In some embodiments, the method comprises measuring a distance from a catheter to the object. In some embodiments, the distance from the catheter to the object is displayed in real time with a visual representation. In some embodiments, a fiducial location in the extravascular data is a feature not shown in the intravascular data. In some embodiments, the fiducial location comprises a radiopaque marker of a catheter. In some embodiments, the fiducial location comprises a known correlation to the intravascular data. In some embodiments, the known correlation comprises a distance. In some embodiments, the first object or the second object are displayed superimposed on the real-time extravascular data in one or more data views. In some embodiments, a first view of the one or more data views comprises a display of the real-time extravascular data without the display of the first object or the second object, and where a second view of the one or more data views comprises a display of the real-time extravascular data with the display of the first object or the second object. In some embodiments, a view of the one or more data views comprises a zoom view. In some embodiments, displaying the first object or the second object relative to the location within the intravascular data or the location within the extravascular data comprises a first state where the display of the first object or the second object is visible, or a second state where the display of the first object or the second object is not visible. In some embodiments, the` display of the first object or the second object superimposed on the real-time extravascular data is displayed on one or more monitors. In some embodiments, the one or more monitors comprise an internal monitor positioned to face an operator of medical equipment, external monitor positioned to face medical personnel using the medical equipment, or any combination thereof configurations. In some embodiments, the internal monitor and the external monitor comprise different view configurations. In some embodiments, the one or more monitors comprise at least 2 external monitors positioned to face medical personnel using medical equipment, where the at least 2 external monitors comprise different view configurations. In some embodiments, the method comprises displaying an indicator, where the indicator comprises a metric representing a distance between the object and a target location within the real-time extravascular data. In some embodiments, the target location is determined by at least one intravascular image or at least one extravascular image. In some embodiments, the method comprises processing a vessel geometry of the extravascular data and displaying the processed vessel geometry in a view of the one or more data views. In some embodiments, the extravascular data or the intravascular data is acquired without the use of contrast or with variable use of contrast.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1B shows an intravascular imaging system and probe, as described in some embodiments herein.

FIG. 2 shows a flow diagram for a method of registering an intravascular and extravascular dataset, as described in some embodiments herein.

FIGS. 3A-3C show a view configuration with one or more data views displaying the registered intravascular and extravascular data, as described in some embodiments herein.

FIGS. 4A-4D show a flow diagram for a method of guiding an intravascular device through a blood vessel by referencing registered intravascular and extravascular data, as described in some embodiments herein.

FIG. 5 shows a flow diagram for a method of landmark guidance of an intravascular device shown by a vessel cross-sectional view of extravascular data registered to intravascular data, as described in some embodiments herein.

FIG. 6 shows a flow diagram of a method of processing non-linear shaped extravascular vessel data to a straight line, as described in some embodiments herein.

FIGS. 7A-7C show one or more views of one or more user interfaces, as described in some embodiments herein.

FIGS. 8A-8B show one or more data views displaying various perspectives of extravascular data and landmark overlays, as described in some embodiments herein.

FIGS. 9A-9B show one or more views of a first monitor and a second monitor displaying extravascular data and intravascular landmark overlay, as described in some embodiments herein.

FIG. 10 shows a view of a user interface displaying a combination of extravascular data and intravascular landmarks registered to the extravascular data in one or more data views, as described in some embodiments herein.

FIG. 11 shows a diagram of a computer system configured to conduct the methods of the disclosure, as described in some embodiments herein.

FIG. 12 shows a workflow diagram of a method of registering extravascular data and intravascular data in real-time without the use of contrast or with variable use of contrast, as described in some embodiments herein.

FIGS. 13A-13G show extravascular data processed and/or analyzed for objects and/or landmarks in real-time that are used to register intravascular data to real-time extravascular data, as described in some embodiments herein.

FIG. 14 shows a workflow diagram of a method of correcting, shifting, and/or adjusting a position and/or location of intravascular data registered to extravascular data without the use of contrast or with variable use of contrast, as described in some embodiments herein.

FIGS. 15A-15D show intravascular data registered to real-time extravascular data and the adjustment of the registered intravascular data location over time as the registered extravascular data location shift due to patient motion, as described in some embodiments herein.

FIG. 16 shows a workflow diagram of a method of guiding and/or implanting an object at an indicator of the one or more locations in registered extravascular and/or intravascular dataset collected and/or acquired without the use of contrast overlaid on real-time extravascular data with contrast (e.g., variable contrast), as described in some embodiments herein.

FIG. 17 shows a workflow diagram of a method of guiding and/or implanting an object at an indicator of the one or more locations in a registered extravascular and/or intravascular dataset collected and/or acquired with contrast overlaid on real-time extravascular data without contrast, as described in some embodiments herein.

FIG. 18 shows a workflow diagram of a method of guiding and/or implanting an object at an indicator of the one or more locations in a registered extravascular and/or intravascular dataset collected and/or acquired without contrast overlaid on real-time extravascular data without contrast, as described in some embodiments herein.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

Considering and/or evaluating only one of intravascular or extravascular data would not render a complete representation of the complex biological system of blood vessels and how to treat them. For example, x-ray angiography has been shown to be a useful tool for rapidly assessing the contour and macroscopic morphology of blood vessels to determine a stenotic vessel requiring stenting, and for real-time guidance of vessel treatment. However, the data representation of x-ray angiography lacks biochemical (e.g., the type of plaque or composition of the plaque) or microscopic anatomical characterization (e.g., thin cap fiber atheroma structure of vulnerable plaques) of a blood vessel. The combination of intravascular and extravascular data of blood vessels, as described by the systems and methods herein, may reduce procedure time of image-guided (e.g., fluoroscopy-guided) interventions (e.g., percutaneous coronary intervention and/or stent placement) and may increase the accuracy of placement and/or guidance of medical devices (e.g., stents, catheters, ablation devices), ultimately increasing efficacy of treatment. In some cases, the accuracy may increase by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 96%, at least about 97%, at least about 98%, or at least about 99%.

In a typical intravascular blood vessel imaging procedure (e.g., intravascular ultrasound and/or intravascular optical coherence tomography), a region of a blood vessel is navigated to under x-ray fluoroscopy e.g., through use of a radio-opaque marker positioned relative to an imaging probe inserted into the blood vessel. Once the imaging probe has been guided to a region of interest, the imaging probe then collects volumetric data of the blood vessel and is subsequently removed from the individual. Unfortunately, the rich dataset of the intravascular imaging dataset on its own without co-registration to the position of the dataset within the extravascular dataset limits actionable insight for medical care personnel. The systems and methods described herein provide a solution of registering and/or combining the intravascular and extravascular dataset to realize the unexpected benefit of a registration between the two datasets (e.g., during live image-based guidance).

The disclosure provided herein describes methods and systems of acquiring, correlating, registering, and/or displaying extravascular and intravascular data, (e.g., image data, catheter pressure, spatial position of a catheter, etc.) acquired during intravascular and/or extravascular procedures. In some cases, the methods and/or systems of acquiring, correlating, registering, and/or displaying extravascular and intravascular data may be conducted and/or operated without the use of a contrast agent. In some cases, the methods and/or systems of acquiring, correlating, registering, and/or displaying extravascular and intravascular data may be conducted and/or operated with the use of variable contrast, as described elsewhere herein. In some cases, the variable use of contrast may comprise injecting and/or providing up to about 1 second or up to about 2 seconds of contrast agent to a subject’s vascular network. In some cases, the contrast may be provided at least once, at least twice, or at least three times during intravascular and/or extravascular data collection, as described elsewhere herein. In some cases, the extravascular and intravascular data may be registered, acquired, correlated, and/or displayed in real-time. In some cases, real-time data registration, acquisition, correlation, and/or display may be completed at a real-time data rate. In some cases, the real-time data rate may comprise a frequency of about 25 Hz to about 120 Hz. In some cases, the real-time data rate may comprise about 25 Hz to about 30 Hz, about 25 Hz to about 35 Hz, about 25 Hz to about 40 Hz, about 25 Hz to about 45 Hz, about 25 Hz to about 50 Hz, about 25 Hz to about 55 Hz, about 25 Hz to about 60 Hz, about 25 Hz to about 70 Hz, about 25 Hz to about 80 Hz, about 25 Hz to about 100 Hz, about 25 Hz to about 120 Hz, about 30 Hz to about 35 Hz, about 30 Hz to about 40 Hz, about 30 Hz to about 45 Hz, about 30 Hz to about 50 Hz, about 30 Hz to about 55 Hz, about 30 Hz to about 60 Hz, about 30 Hz to about 70 Hz, about 30 Hz to about 80 Hz, about 30 Hz to about 100 Hz, about 30 Hz to about 120 Hz, about 35 Hz to about 40 Hz, about 35 Hz to about 45 Hz, about 35 Hz to about 50 Hz, about 35 Hz to about 55 Hz, about 35 Hz to about 60 Hz, about 35 Hz to about 70 Hz, about 35 Hz to about 80 Hz, about 35 Hz to about 100 Hz, about 35 Hz to about 120 Hz, about 40 Hz to about 45 Hz, about 40 Hz to about 50 Hz, about 40 Hz to about 55 Hz, about 40 Hz to about 60 Hz, about 40 Hz to about 70 Hz, about 40 Hz to about 80 Hz, about 40 Hz to about 100 Hz, about 40 Hz to about 120 Hz, about 45 Hz to about 50 Hz, about 45 Hz to about 55 Hz, about 45 Hz to about 60 Hz, about 45 Hz to about 70 Hz, about 45 Hz to about 80 Hz, about 45 Hz to about 100 Hz, about 45 Hz to about 120 Hz, about 50 Hz to about 55 Hz, about 50 Hz to about 60 Hz, about 50 Hz to about 70 Hz, about 50 Hz to about 80 Hz, about 50 Hz to about 100 Hz, about 50 Hz to about 120 Hz, about 55 Hz to about 60 Hz, about 55 Hz to about 70 Hz, about 55 Hz to about 80 Hz, about 55 Hz to about 100 Hz, about 55 Hz to about 120 Hz, about 60 Hz to about 70 Hz, about 60 Hz to about 80 Hz, about 60 Hz to about 100 Hz, about 60 Hz to about 120 Hz, about 70 Hz to about 80 Hz, about 70 Hz to about 100 Hz, about 70 Hz to about 120 Hz, about 80 Hz to about 100 Hz, about 80 Hz to about 120 Hz, or about 100 Hz to about 120 Hz. In some cases, the real-time data rate may comprise about 25 Hz, about 30 Hz, about 35 Hz, about 40 Hz, about 45 Hz, about 50 Hz, about 55 Hz, about 60 Hz, about 70 Hz, about 80 Hz, about 100 Hz, or about 120 Hz. In some cases, the real-time data rate may comprise at least about 25 Hz, about 30 Hz, about 35 Hz, about 40 Hz, about 45 Hz, about 50 Hz, about 55 Hz, about 60 Hz, about 70 Hz, about 80 Hz, or about 100 Hz. In some cases, the real-time data rate may comprise at most about 30 Hz, about 35 Hz, about 40 Hz, about 45 Hz, about 50 Hz, about 55 Hz, about 60 Hz, about 70 Hz, about 80 Hz, about 100 Hz, or about 120 Hz.

In some cases, the real-time data rate may comprise a real-time imaging frequency when e.g., acquiring and/or displaying intravascular and/or extravascular image data. In some instances, extravascular data and/or intravascular data may be displayed at real-time imaging frequencies. In some cases, real time imaging frequencies may comprise at least about 30 imaging frames of e.g., intravascular and/or extravascular data, displayed and/or acquired per second. In some cases, real-time imaging frequency may comprise about 25 frames per second (fps) to about 120 fps. In some cases, real-time imaging frequency may comprise about 25 fps to about 30 fps, about 25 fps to about 35 fps, about 25 fps to about 40 fps, about 25 fps to about 45 fps, about 25 fps to about 50 fps, about 25 fps to about 55 fps, about 25 fps to about 60 fps, about 25 fps to about 70 fps, about 25 fps to about 80 fps, about 25 fps to about 100 fps, about 25 fps to about 120 fps, about 30 fps to about 35 fps, about 30 fps to about 40 fps, about 30 fps to about 45 fps, about 30 fps to about 50 fps, about 30 fps to about 55 fps, about 30 fps to about 60 fps, about 30 fps to about 70 fps, about 30 fps to about 80 fps, about 30 fps to about 100 fps, about 30 fps to about 120 fps, about 35 fps to about 40 fps, about 35 fps to about 45 fps, about 35 fps to about 50 fps, about 35 fps to about 55 fps, about 35 fps to about 60 fps, about 35 fps to about 70 fps, about 35 fps to about 80 fps, about 35 fps to about 100 fps, about 35 fps to about 120 fps, about 40 fps to about 45 fps, about 40 fps to about 50 fps, about 40 fps to about 55 fps, about 40 fps to about 60 fps, about 40 fps to about 70 fps, about 40 fps to about 80 fps, about 40 fps to about 100 fps, about 40 fps to about 120 fps, about 45 fps to about 50 fps, about 45 fps to about 55 fps, about 45 fps to about 60 fps, about 45 fps to about 70 fps, about 45 fps to about 80 fps, about 45 fps to about 100 fps, about 45 fps to about 120 fps, about 50 fps to about 55 fps, about 50 fps to about 60 fps, about 50 fps to about 70 fps, about 50 fps to about 80 fps, about 50 fps to about 100 fps, about 50 fps to about 120 fps, about 55 fps to about 60 fps, about 55 fps to about 70 fps, about 55 fps to about 80 fps, about 55 fps to about 100 fps, about 55 fps to about 120 fps, about 60 fps to about 70 fps, about 60 fps to about 80 fps, about 60 fps to about 100 fps, about 60 fps to about 120 fps, about 70 fps to about 80 fps, about 70 fps to about 100 fps, about 70 fps to about 120 fps, about 80 fps to about 100 fps, about 80 fps to about 120 fps, or about 100 fps to about 120 fps. In some cases, real-time imaging frequency may comprise about 25 fps, about 30 fps, about 35 fps, about 40 fps, about 45 fps, about 50 fps, about 55 fps, about 60 fps, about 70 fps, about 80 fps, about 100 fps, or about 120 fps. In some cases, real-time imaging frequency may comprise at least about 25 fps, about 30 fps, about 35 fps, about 40 fps, about 45 fps, about 50 fps, about 55 fps, about 60 fps, about 70 fps, about 80 fps, or about 100 fps. In some cases, real-time imaging speed may comprise at most about 30 fps, about 35 fps, about 40 fps, about 45 fps, about 50 fps, about 55 fps, about 60 fps, about 70 fps, about 80 fps, about 100 fps, or about 120 fps.

In some instances, the real-time data rate and/or the real-time imaging frequency may reduce imaging artifacts and/or noise (e.g., breathing of the subject, motion of the subject), as described elsewhere herein, by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% in comparison to devices, methods, and/or systems operating at less than a real-time data rate. In some cases, the real-time data rate and/or the real-time imaging frequency may increase an accuracy of guiding a device through a blood vessel and/or placing of a device within a blood vessel at a region and/or location, as described elsewhere herein, by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% in comparison to devices, methods, and/or systems operating at less than real-time data rate and/or real-time imaging frequencies.

In some cases, extravascular imaging may comprise x-ray angiography with or without contrast and/or magnetic resonance imaging (MRI). In some cases, intravascular imaging may comprise optical coherence tomography, light endoscopy, ultrasound, near infrared spectroscopy, photoacoustic tomography, light endoscopy, fluorescence, or any combination thereof. Each imaging modality alone provides a particular data type e.g., macroscopic vessel structure or microscopic vessel structure that one imaging modality alone cannot solely provide.

In some cases, the systems described elsewhere herein may e.g., acquire intravascular data by the systems and/or devices, described elsewhere herein, that may be annotated or marked by an object that may then be registered to intravascular data, extravascular data, or a combination thereof. The object (e.g., a landmark), set from the perspective of the intravascular data may be visualized e.g., superimposed on a corresponding region in an extravascular dataset. In some cases, the visualization of the object may be superimposed on a real-time acquisition of extravascular data. In some instances, the position of the object and/or landmark may be dynamically adjusted based on the movement and/or motion artifact e.g., breathing, micro-tremors, etc. of a subject and/or patient, when displayed superimposed on extravascular data. In some cases, the movement and/or motion artifact of the extravascular data e.g., due to breathing, micro-tremors, etc. of the patient may be removed from the extravascular data. In some instances, removing movement and/or motion artifact of the extravascular data may increase the accuracy of a position of the registered object with respect to the intravascular, extravascular, or any combination thereof data. In some cases, the increase in accuracy may comprise at least about 1%, at least about 2%, at least about 3%, at least about 4%, at least about 5%, at least about 10%, at least about 15%, at least about 20%, at least about 25% at least about 50%, at least about 60%, at least about 70%, at least about 80%, at least about 90%, at least about 95%, or at least about 99% increase in accuracy compared to an accuracy of the position of the registered object without removing the movement and/or motion artifact of the extravascular data.

In some cases, the methods and/or systems of acquiring, correlating, registering, and/or displaying extravascular and intravascular data, described elsewhere herein, may be conducted, performed, and/or used, without a contrast agent. Pre-existing techniques of extravascular data collection, e.g., fluoroscopic angiography, require the use of an iodine-based contrast agent to visualize a vascular network and the fluid dynamics of blood through e.g., the coronary artery prior to, during, and/or after placement of an intravascular device (e.g., a cardiovascular sent). Prolonged or repeated flushing of Iodine through the vascular network has been found, in some cases, to cause or bring about allergic reactions or hyperthyroidism for some patients, as well the accrual of damage to kidneys and sometimes acute and chronic kidney injury. Therefore, the minimization of contrast usage during diagnosis and treatment of a patient is desired for optimal patient outcomes. The disclosure provided herein describes method and systems for correlating and/or registering intravascular data to extravascular data with, without the use of a contrast agent, or with variable use of a contrast agent when acquiring extravascular data or registering and/or correlating extravascular data and intravascular in real-time.

### Imaging System

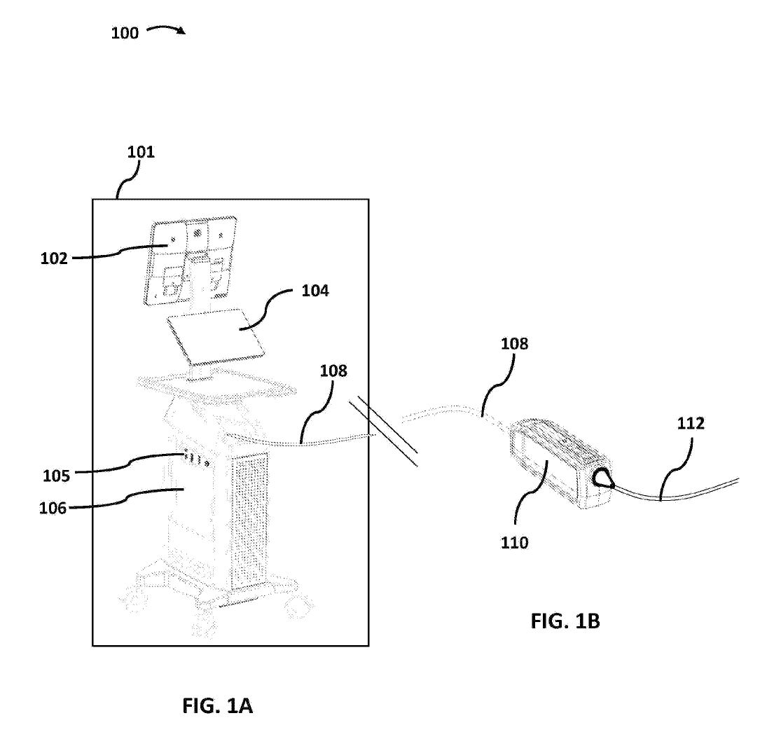

The disclosure provided herein describes a system 100 that registers intravascular and extravascular data, as seen in FIG. 1. The system 100 may comprise an imaging system 101 and display, shown in FIG. 1A, configured to acquire intravascular data and register the intravascular data with extravascular data. In some instances, the intravascular data may comprise one or more intravascular images of a blood vessel. In some cases, the extravascular data may comprise one or more extravascular images (e.g., x-ray angiogram, MRI, etc.) of blood vessel shape, physiology, anatomy, or any combination thereof. In some cases, the imaging system may comprise a computer system (106, 1110) to process intravascular, extravascular, user interaction, or any combination thereof data.

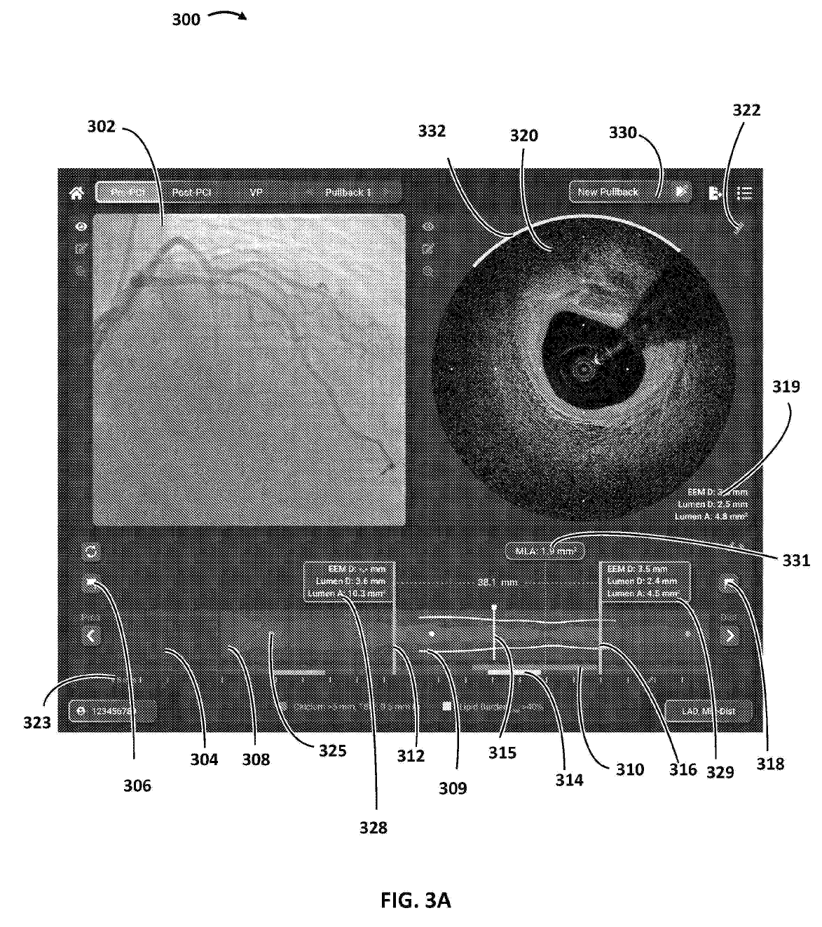

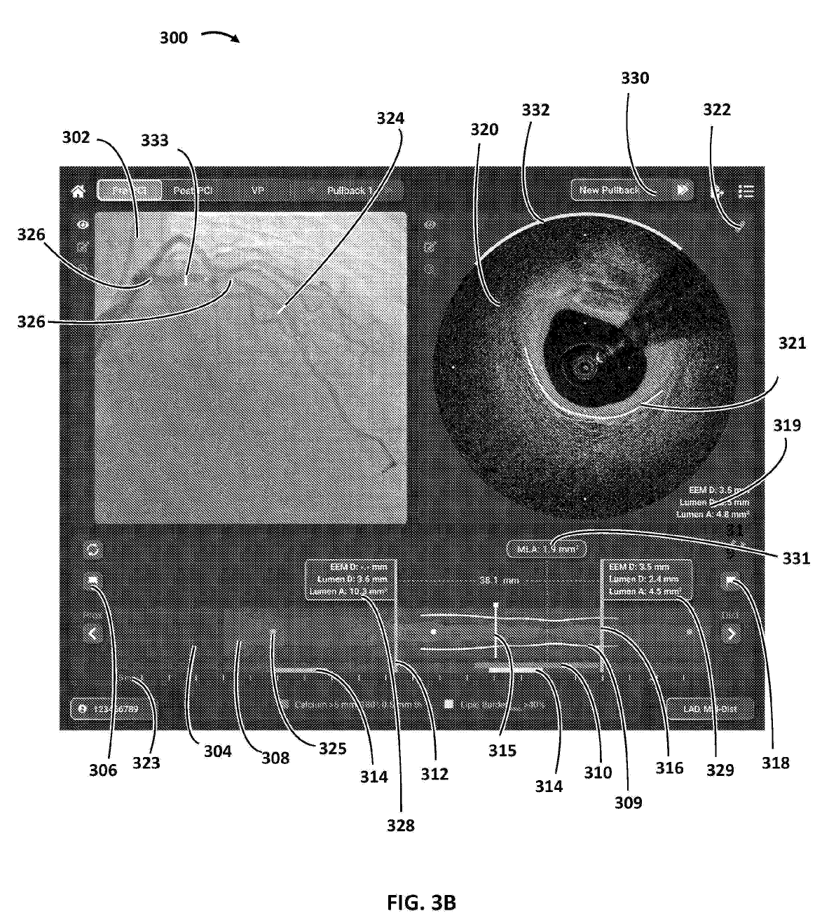

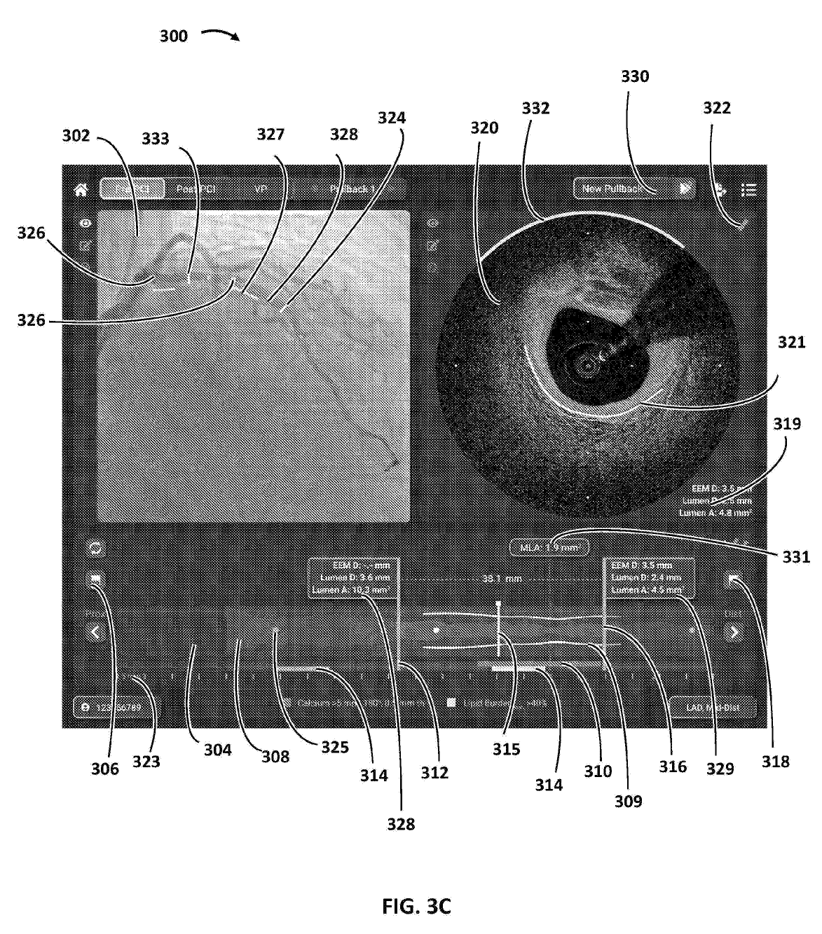

The user interaction data may comprise a user inputting data into the imaging system 101 where the data may comprise patient information, landmark designation, selecting system operation modes, image processing functions, or any combination thereof. In some cases, a user may input data into the imaging system 101 with a mouse and/or keyboard electrically coupled with the computer system (106, 1110). A user may visualize a view configured (i.e., user interface) to input data into the system via a first monitor 102 and/or a second monitor 104. In some cases, the first monitor 102 and/or the second monitor 104 may comprise a touchscreen interface and keyboard for interacting, acquiring, or any combination thereof actions conducted on the intravascular and/or extravascular data. In some cases, the user interaction data may comprise data resulting from a user interacting with the extravascular and/or intravascular data (e.g., rotating, zooming in, adjusting contrast, adjusting brightness, measuring a distance, etc.). The computer system (106, 1110) may include or be in communication with an electronic display 1114 (e.g., the first monitor 102 and/or the second monitor 104) that comprises one or more view configurations (i.e., user interface (UI)) 1116, as also shown in FIGS. 3A-10, described elsewhere herein, for viewing the intravascular data, extravascular data, a registered and/or combination of the intravascular and extravascular data, or any combination thereof.

In some cases, the computer system (106, 1110) may comprise an input interface 105, where the input interface 105 may comprise one or more input points and/or ports electrically coupled with the computer system (106, 1110). The input interface 105 may receive one or more data and/or streams of data from one or more imaging systems. For example, the input interface 105 may receive an x-ray angiography data, where the computer system (106, 1110) may then register the x-ray angiography data with the intravascular data. In some cases, the input interface 105 may receive angiography-derived physiology, MRI, computed tomography, spatial positional, intravascular sensor (e.g., intravascular physiology), or any combination thereof data from one or more medical devices to be displayed and/or registered to the intravascular data. In some instances, the input interface 105, may receive the data to register to the extravascular data, as described elsewhere herein, wirelessly through an ad-hoc WIFI, Bluetooth, radiofrequency, or any combination thereof wireless communication platform.

In some cases, the computer system (106, 1110) may process data with one or more processors 1104, described elsewhere herein. In some instances, the one or more processors may comprise processors of one or more graphical processing units, integrated circuit, or any combination thereof processors. The graphical processing units provide the capability of processing complex large datasets due to their highly parallel processor architecture. For example, processing data with one or more graphical processing units provides the system with the capability of registering the intravascular data with a real-time stream of extravascular data, otherwise not achieved with traditional multi-core processors.

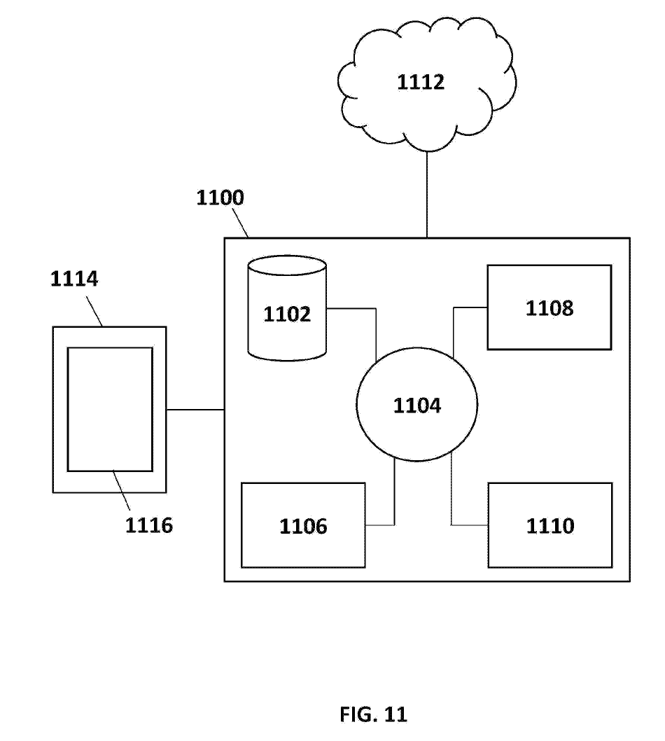

In some cases, the computer system (106, 1110) may be configured to process the intravascular and extravascular data and/or images. The computer system (1100, 106) as seen in FIG. 11, may comprise a central processing unit and/or graphical processing (CPU and/or GPU, also “processor” and “computer processor” herein) 1104, which may be a single core or multi core processor, or a plurality of processor for parallel processing. The computer system (106, 1110) may further comprise memory or memory locations 1106 (e.g., random-access memory, read-only memory, flash memory), electronic storage unit 1102 (e.g., hard disk), communications interface 1108 (e.g., network adapter) for communicating with one or more other devices, and peripheral devices 1110, such as cache, other memory, data storage and/or electronic display adapters. The memory 1106, storage unit 1102, communications interface 1108, and peripheral devices (e.g., mouse, keyboard, etc.) 1110 may be in communication with the CPU and/or GPU 1104 through a communication bus (solid lines), such as a motherboard. The storage unit 1102 may be a data storage unit (or a data repository) for storing data. The computer system (106, 1110) may be operatively coupled to a computer network (“network”) 1112 with the aid of the communication interface 1108. The network 1112 may be the Internet, an internet and/or extranet, or an intranet and/or extranet that is in communication with the Internet. The network 1112 may, in some cases, be a telecommunication and/or data network. The network 1112 may include one or more computer servers, which may enable distributed computing, such as cloud computing. The network 1112, in some cases with the aid of the computer system (106, 1110), may implement a peer-to-peer network, which may enable devices coupled to the computer system (106, 1110) to behave as a client or a server.

The CPU and/or GPU 1104 may execute a sequence of machine-readable instructions, which may be embodied in a program or software. The instructions may be directed to the CPU and/or GPU 1104, which may subsequently program or otherwise configured the CPU/GPU 1104 to acquire data and/or process data produced by the imaging system described elsewhere herein. In some embodiments, the computer system (106, 1110) central processing unit and/or graphical processing unit 1104 may execute machine executable or machine-readable code that may be provided in the form of software to transfer data generated by the imaging system to a network and/or cloud 1112 for further processing, classification, data clustering, or any combination thereof operations. In some instances, the data may comprise the intravascular and/or extravascular data, described elsewhere herein. In some cases, the data may comprise image pixel data. In some instances, the pixel data may comprise optical coherence tomography, x-ray angiography, computed tomography, intravascular ultrasound, spectroscopy, MRI, or any combination thereof image pixel data.

In some embodiments, the CPU and/or GPU 1104 may be part of a circuit, such as an integrated circuit. One or more other components of the system 1110 may be included in the circuit. In some cases, the circuit may comprise an application specific integrated circuit (ASIC). The storage unit 1102 may store files, such as drivers, libraries, and saved programs. The storage unit 1102 may store acquired x-ray angiography, optical coherence tomography, intravascular ultrasound, near infrared spectroscopy, photoacoustic or any combination thereof data and/or images. In some cases, the intravascular and/or extravascular data and/or images may be stored in the cloud, a medical system electronic medical records (e.g., EPIC), or any combination thereof locations. The computer system (106, 1110), in some cases may comprise one or more additional data storage units that are external to the computer system (106, 1110), such as located on a remote server that is in communication with the computer system (106, 1110) through an intranet or the internet 1112.

In some cases, the imaging system 101 is in electrical and/or optical communication to an imaging probe actuator 110, and an imaging probe 112, as seen in FIG. 1B. The imaging system 101 may be in electrical and/or optical communication with the imaging probe actuator 110 through one or more electrical and/or optical communication wires 108. In some cases, the imaging probe 112 may be releasably coupled to the imaging probe actuator 110, such that a first imaging probe may be removed from the imaging probe actuator and replaced with a second imaging probe.

In some instances, the imaging probe may comprise an intravascular imaging probe. The intravascular imaging probe may comprise an optical coherence tomography, intravascular ultrasound, reflectance, photoacoustic, near infrared spectroscopy, fluorescence, or any combination thereof imaging probes. In some instances, the imaging probe may obtain, collected, and/or detect intravascular data from an inner lumen and/or body of a blood vessel. In some cases, the intravascular data may comprise two-dimensional (e.g., circular cross-sectional data), and/or volumetric intravascular data (i.e., one or more two-dimensional circular cross-sectioned data as a function of the length of the optical axis of the imaging probe). In some cases, the imaging probe may comprise one or more radio-opaque markers and/or indicia that may be visualized on extravascular imaging modalities e.g., x-ray angiography, computed tomography, MRI, or any combination thereof extravascular imaging modalities.

In some instances, the imaging probe actuator 110 may rotate and/or translate the imaging probe 112, to obtain two and/or three-dimensional intravascular datasets. In some cases, the probe may be rotated by a stepper motor, dc-brushless motor, or any combination thereof motors coupled to an optic rotary joint. In some cases, the imaging probe actuator 110 may translate the imaging probe 112 with a stage, where the stage may comprise a linear and/or a planar translational stage. The stage translation and the rotation of the imaging probe actuator 110 may be set and/or adjusted by a user via the one or more interfaces of the imaging system 101, described elsewhere herein. In some instances, the stage translation and the rotation of the imaging probe actuator 110 may be determine and/or set by the system based on pre-set standard values for a particular type of imaging procedure or frequently used settings.

Aspects of the systems and methods provided herein, such as the computer system (106, 1110), may be embodied in programming. Various aspects of the technology may be thought of a “product” or “articles of manufacture” typically in the form of a machine (or processor) executable code and/or associated data that is carried on or embodied in a type of machine-readable medium. Machine-executable code may be stored on an electronic storage unit, such as memory (e.g., read-only memory, random-access memory, flash memory) or a hard disk. “Storage” type media may include any or all of the tangible memory of a computer, processor the like, or associated modules thereof, such as various semiconductor memories, tape drives, disk drives and the like, which may provide non-transitory storage at any time for the software program. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage’ media, term such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media may include, for example, optical or magnetic disks, such as any of the storage devices in any computer(s) or the like, such as may be used to implement the databases, etc. Volatile storage media may include dynamic memory, such as main memory of such a computer platform. Tangible transmission media includes coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instruction to a processor for execution.

### Computer Systems and Machine Learning Models

In some embodiments, the system 100 disclosed herein may comprise a computer system (106, 1110) suitable for implementing machine learning algorithms and/or predictive models configured to analyze, process, segment and/or label extravascular and/or intravascular data collected by the imaging system 101, imaging probe 112, and imaging probe actuator 110 described elsewhere herein. In some cases, one or more intravascular and/or extravascular images may be generated from the intravascular and/or extravascular data. In some cases, predictive models e.g., machine learning models and/or machine learning algorithms may analyze, extract, condense, reduce, predict, process, classify, segment or any combination thereof operations conducted on the intravascular and/or extravascular data.

In some embodiments, the systems disclosed herein may implement one or more machine learning algorithms and/or model(s) to identify, classify, process and/or segment regions of interest of intravascular and/or extravascular data. In some embodiments, the systems disclosed herein may implement one or more machine learning algorithms to register one or more images of a first extravascular data to one or more reference images, or to one or more images of a second extravascular data. In some cases, the first extravascular data may be the same as the second extravascular data. In some instances, the first extravascular data may be different than the second extravascular data. For example, a machine learning algorithm may be trained with labeled intravascular and/or extravascular data such that when provided an input of unlabeled intravascular and/or extravascular data, the machine learning algorithm may classify each data point into one or more categories and/or features. In some cases, each data point may comprise a pixel or a plurality of pixels of the intravascular and/or extravascular data. In some instances, intravascular and/or extravascular data may be labeled by a user on the system. The labeled data may then be used to train one or more machine learning models on the system and/or within a remote cloud-based computing architecture. The remote cloud-based computing architecture may be improved by one or more systems through a wireless communication platform (i.e., WIFI). In some embodiments, a human user may select, and discard features prior/during machine learning training/classification. In some cases, a computer may select and discard features. In some cases, the features may be discarded based on a threshold value.

In some instances, the one or more categories and/or features of the labeled data may then be provided to one or more treatment parameter machine learning model and/or algorithms to determine suggested treatment and/or treatment parameters (e.g., what type of stent to place and where spatially to best place the stent to achieve clinical efficacy of treatment). The one or more treatment parameter machine learning models may be trained with prior features and corresponding treatment efficacy (i.e., whether any complications ensued after clinical intervention with the system) to generate one or more trained treatment parameter machine learning models to predict efficacious treatments. The spatial orientation of labeled features and their relationship to one another may be other features determined and considered by the treatment parameter machine learning models.

In some cases, the one or more categories and/or features of data for extravascular data may comprise background data, healthy blood vessel morphology, stenotic blood vessel morphology, or occluded blood vessel. In some cases, the one or more categories of data for the intravascular data may comprise blood vessel tissue of the epithelium, blood vessel tissue of the intima, blood vessel tissue of the adventitia, plaque within the blood vessel tissue, vulnerable plaque within the blood vessel tissue, or any combination thereof. In some cases, the one or more categories and/or features of intravascular data may comprise spectroscopic (e.g., in the near infrared) signature of the intravascular blood vessel tissue. For example, the one or more categories and/or features may classify the composition of plaque of the blood vessel based on its spectroscopic signature. In some cases, the one or more categories may comprise a calcium or a lipid spectroscopic signature. In some instances, the machine learning model and/or algorithm may pre-process the intravascular and/or extravascular data prior to classifying a feature of the data. In some instances, prep-processing the intravascular and/or extravascular data may comprise de-noising, smoothening, averaging, sharpening, brightness and/or contrast adjustment, or any combination thereof mathematical manipulation of the data. In some cases, the features and/or categories of the intravascular and/or extravascular data may be extracted without a pre-processing step.

In some cases, machine learning algorithms may need to extract and draw relationships between features as conventional statistical techniques may not be sufficient. In some cases, machine learning algorithms may be used in conjunction with conventional statistical techniques. In some cases, conventional statistical techniques may provide the machine learning algorithm with pre-processed features.

In some embodiments, any number of features may be classified by the machine learning algorithm. The machine learning algorithm may classify at least 1 feature. In some cases, the plurality of features may include between about 1 feature to 5 features. In some cases, the plurality of features may include between about 5 features to 10 features. In some cases, the plurality of features may include between about 10 features to 50 features.

In some embodiments, the machine learning algorithm may be, for example, an unsupervised learning algorithm, supervised learning algorithm, or a combination thereof. The unsupervised learning algorithm may be, for example, clustering, hierarchical clustering, k-means, mixture models, DBSCAN, OPTICS algorithm, VoxelMorph algorithm, anomaly detection, local outlier factor, neural networks, autoencoders, deep belief nets, hebbian learning, generative adversarial networks, self-organizing map, expectation–maximization algorithm (EM), method of moments, blind signal separation techniques, principal component analysis, independent component analysis, non-negative matrix factorization, singular value decomposition, or a combination thereof. The supervised learning algorithm may be, for example, support vector machines, linear regression, logistic regression, linear discriminant analysis, decision trees, k-nearest neighbor algorithm, neural networks, similarity learning, or a combination thereof. In some embodiments, the machine learning algorithm may comprise a deep neural network (DNN). The deep neural network may comprise a convolutional neural network (CNN). The CNN may be, for example, U-Net, ImageNet, LeNet-5, AlexNet, ZFNet, GoogleNet, VGGNet, ResNet18 or ResNet, etc. Other neural networks may be, for example, deep feed forward neural network, recurrent neural network, LSTM (Long Short-Term Memory), GRU (Gated Recurrent Unit), Auto Encoder, variational autoencoder, adversarial autoencoder, denoising auto encoder, sparse auto encoder, Boltzmann machine, RBM (Restricted BM), deep belief network, generative adversarial network (GAN), deep residual network, capsule network, or attention/transformer networks, etc.

In some instances, the machine learning model may comprise clustering, scalar vector machines, kernel SVM, linear discriminant analysis, Quadratic discriminant analysis, neighborhood component analysis, manifold learning, convolutional neural networks, reinforcement learning, random forest, Naive Bayes, gaussian mixtures, Hidden Markov model, Monte Carlo, restrict Boltzmann machine, linear regression, or any combination thereof.

In some cases, the machine learning algorithm may include ensemble learning algorithms such as bagging, boosting and stacking. The machine learning algorithm may be individually applied to the plurality of features extracted.

In some embodiments, the systems may apply one or more machine learning algorithms and/or an ensemble of machine learning algorithms.

In some embodiments, the machine learning algorithm may have a variety of parameters. The variety of parameters may be, for example, learning rate, minibatch size, number of epochs to train for, momentum, learning weight decay, or neural network layers etc.

In some embodiments, the learning rate may be between about 0.00001 to 0.1.

In some embodiments, the minibatch size may be at between about 16 to 128.

In some embodiments, the neural network may comprise neural network layers. The neural network may have at least about 2 to 1000 or more neural network layers.

In some embodiments, the number of epochs to train for may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some embodiments, the momentum may be at least about 0.1, 0.2, 0.3, 0.4, 0.5, 0.6, 0.7, 0.8, 0.9 or more. In some embodiments, the momentum may be at most about 0.9, 0.8, 0.7, 0.6, 0.5, 0.4, 0.3, 0.2, 0.1, or less.

In some embodiments, learning weight decay may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some embodiments, the learning weight decay may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some embodiments, the machine learning algorithm may use a loss function. The loss function may be, for example, regression losses, mean absolute error, mean bias error, hinge loss, Adam optimizer and/or cross entropy.

In some embodiments, the parameters of the machine learning algorithm may be adjusted with the aid of a human and/or computer system.

In some embodiments, the treatment parameter machine learning model and/or algorithms may prioritize certain features. The treatment parameter machine learning model and/or algorithms may prioritize features that may be more relevant for determining anatomical and/or physiologic features to characterize variation in blood vessel geometry and composition. In some cases, the blood vessel geometry and composition may classify a portion of a blood vessel as diseased (e.g., thin cap fiber atheroma, vulnerable plaque, stabile plaque, etc.). In some cases, the features may be prioritized using a weighting system. In some cases, the features may be prioritized on probability statistics based on the frequency and/or quantity of occurrence of the feature. The machine learning algorithm may prioritize features with the aid of a human and/or computer system.

In some embodiments, one or more of the features may be used with machine learning or conventional statistical techniques to determine if a segment of intravascular and/or extravascular data is likely to contain artifacts. The identified artifacts may be a result of optical misalignment, movement of the subject during intravascular and/or extravascular data acquisition, laser power instability, laser pulse frequency jitter, movement of the subject via breathing or micro-tremors, or any combination thereof artifact. In some cases, movement sensors or other sensors may be used as an additional input to the artifact reduction machine learning model and/or algorithm. In some cases, the identified artifacts can be rejected from being used in blood vessel anatomy and/or disease classification.

In some cases, the machine learning algorithm may prioritize certain features to reduce calculation costs, save processing power, save processing time, increase reliability, or decrease random access memory usage, etc.

Methods as described herein may be implemented by way of machine (e.g., computer processor) executable code stored on non-transitory electronic storage medium of the computer system 1110, such as, for example, on the memory 1106 or electronic storage unit 1102. The machine executable or machine-readable code may be provided in the form of software. During use, the code may be executed by the processor (i.e., CPU and/or GPU) 1104. In some instances, the code may be retrieved from the storage unit 1102 and stored on the memory 1106 for ready access by the processor 1104. In some instances, the electronic storage unit 1102 may be precluded, and machine-executable instructions are stored on memory 1106.

The code may be pre-compiled and configured for use with a machine having a processor adapted to execute the code or may be compiled during runtime. The code may be supplied in a programming language that may be selected to enable the code to be executed in a pre-compiled or as-compiled fashion.

### Methods

In some cases, the disclosure provided herein describes methods of registering and/or processing intravascular and extravascular data.

In some cases, the method may comprise a method of displaying an object, comprising: displaying an object relative to a feature of an intravascular dataset or a feature of an extravascular dataset, where the feature of the intravascular dataset and the feature of the extravascular dataset are registered to a real-time extravascular dataset, and where the object is superimposed on a display of the real-time extravascular data.

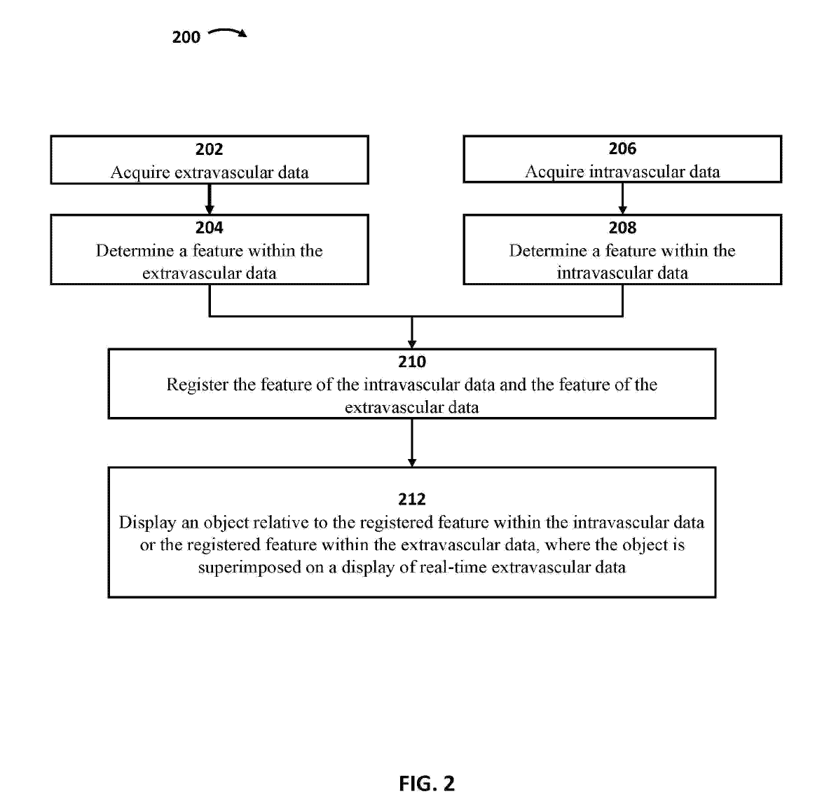

In some cases, the method may comprise a method of displaying an object 200, as seen in FIG. 2. In some instances, the method may comprise the steps of: acquiring extravascular data 202 and intravascular data 206; determining a feature of the extravascular data 204 and a feature of the intravascular data 208; registering the feature of the intravascular data to the feature of the extravascular data 210; and displaying an object relative to the registered feature within the intravascular data or the registered feature within the extravascular data, where the object is superimposed on a display of real-time extravascular data 212. In some instances, the intravascular data comprises at least one image. In some cases, the extravascular data comprises at least one image. In some instances, the extravascular data may comprise x-ray angiogram, computed tomography (CT), magnetic resonance imaging (MRI), ultrasound, fluoroscopy, or any combination, or derivative (e.g., angioFFR, CT-FFR, quantitative coronary angiography, CT-based plaque detection) thereof data.

In some cases, the feature within the intravascular data and the feature within the extravascular data are manually selected (e.g., by a user, medical personal, surgeon, attending physician, nurse, etc.) or are automatically selected by a computer software (e.g., a predictive model, machine learning model and/or algorithm), described elsewhere herein. In some instances, the feature within the intravascular data comprises a location within the intravascular data, and the feature within the extravascular data comprises a location within the extravascular data. In some instances, the object comprises a first object and second object, where the first object and the second object are displayed relative to the registered location of the intravascular data or the registered location of the extravascular data. In some instances, the object may comprise a fiducial marker, where the spatial position of the fiducial marker may be adjusted to account for motion artifact as the real-time extravascular data are displayed.

In some cases, the location within the extravascular data may comprise a location derived from an a ‘prior selection, annotation, or any combination thereof prior patient and/or subject records. In some instances, the method of detecting, registering, and/or displaying an object may further comprise measuring heart cycle data from an external electrocardiogram (ECG) signal, intravascular data, extravascular data, or any combination thereof data. The heart cycle data may be used to improve an accuracy of the registration of the location within the intravascular data and the location within the extravascular data to the real-time extravascular data by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 96%, at least about 97%, at least about 98%, or at least about 99% compared to not measuring heart cycle data. In some cases, the heart cycle data may be used to time the acquisition of any data source (e.g., to improve image quality). In some cases, the method of displaying an object may further comprise displaying an indicator, where the indicator comprises a metric of a distance between the object and a target location within the real-time extravascular data. In some cases, the target location may be determined by at least the intravascular data, at least the extravascular data, or any combination thereof data. In some cases, the method of displaying an object may further comprise processing a vessel geometry of the extravascular data and displaying the processed vessel geometry in a view of one or more data views, described elsewhere herein.

In some cases, the method of displaying an object may further comprise determining a location to guide the positioning of a foreign object. The foreign object may comprise an intravascular stent that may be delivered to a region of the blood vessel through a minimally invasive catheter. In some instances, the method of displaying an object, may further comprise guiding a catheter through a coronary artery to the object to treat coronary artery disease. In some cases, the catheter may comprise an atherectomy catheter. In some instances, the catheter may comprise an imaging probe, as described elsewhere herein. In some instances, the catheter comprises a catheter to measure fractional flow reserve of the coronary artery. In some instances, the registered location of the intravascular data, registered location of the extravascular data, or the location to guide the positioning of a foreign object may comprise a location of: a blood vessel, any representation of blood vessel network, a side-branch of a blood vessel, a region to deploy a stent, a coronary plaque, a guidewire, a guide catheter, a stent, a distal or proximal location of an intravascular imaging pullback, a balloon, a valve, a clip, an atherectomy device, an intravascular data device, or any combination thereof. In some instances, the method of displaying an object may further comprises measuring a distance from the catheter to the object. In some cases, a measured distance from the catheter to the object may be displayed in real time. In some cases, a measured distance from the catheter may be displayed in real time with a visual representation. In some cases, a measured distance from a first detected foreign object to a second detected foreign object may be displayed in real time. In some instances, the measured distance from the first detected foreign object to the second detected foreign object may be displayed in real-time with a visual representation. In some cases, the measured distance from a foreign object to anatomical landmark may be displayed in real time. In some instances, the measured distance from a foreign object to an anatomical landmark may be displayed in real time with a visual representation.

In some cases, the intravascular data may comprise optical coherence tomography (OCT), intravascular ultrasound (IVUS), photoacoustic (PA), near infrared spectroscopy (NIRS), reflectance, Raman spectroscopy, fluorescence, fluorescence lifetime imaging (FLIM), or any combination thereof data. In some instances, the intravascular data may be detected by a multi-modal imaging system (e.g., an OCT-IVUS, or OCT-IVUS-NIRS imaging system). In some instances, the intravascular data may be detected by a one-dimensional sensing system. The one-dimensional sensing system may comprise a pressure sensing system. In some instances, the intravascular data may comprise a measure of flow within a blood vessel.

In some cases, a fiducial location of the extravascular data may comprise a feature that is not shown in the intravascular data. The fiducial location may comprise a radiopaque marker of the catheter or imaging probe, described elsewhere herein. In some instances, the fiducial location may comprise a known correlation to the intravascular data. In some cases, the known correlation may comprise a distance.

In some cases, the real-time extravascular data may be streamed from an x-ray system without transfer of the real-time extravascular data over a network to a processor, described elsewhere herein, configured to display the object superimposed on the display of the real-time extravascular data.

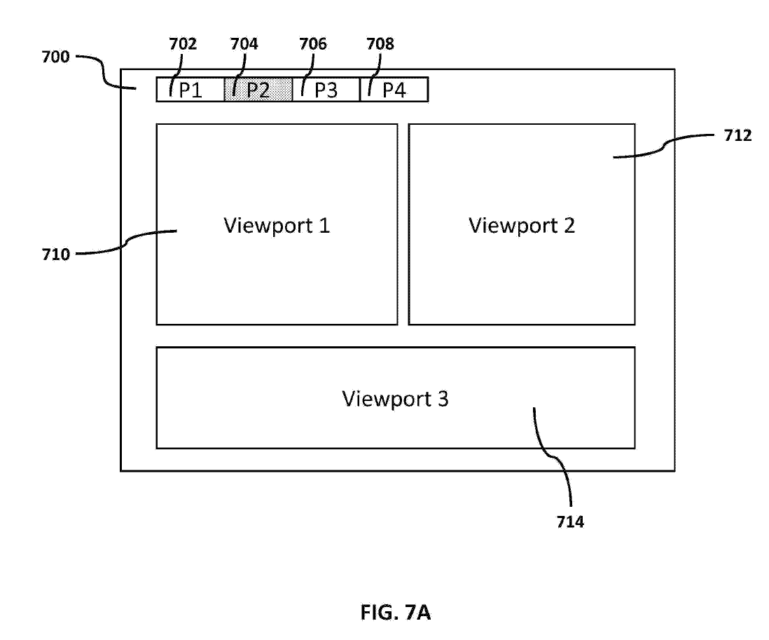

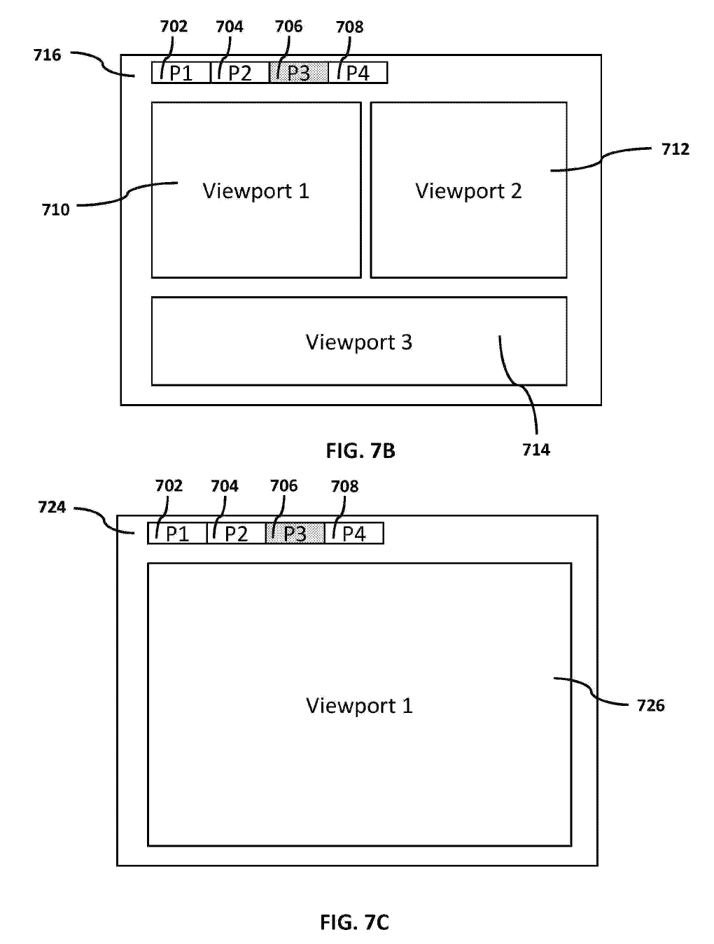

In some instances, the first object and/or the second object may be displayed superimposed on the real-time extravascular data in one or more data views, as shown in FIGS. 7A-10. A first view of the one or more data views may comprise a display of the real-time extravascular data without the display of the first object or the second object. In some cases, a second view of the one or more data views may comprise a display of the real-time extravascular data with display of the first object or the second object. In some instances, a view of the one or more data views may comprise a zoomed view, where the zoom may comprise at least 10%, at least 20%, at least 30%, at least 40%, at least 50%, at least 60%, at least 70%, at least 80%, at least 90%, or at least 100% zoom.

In some cases, displaying the first object or the second object relative to the registered location of the intravascular data or to the registered location of the extravascular data may comprise a first state where the display of the first object or the second object is visible, or a second state where the display of the first object or the second object is not visible. In some cases, the display of the first or the second object superimposed on the real-time extravascular data may be displayed on one or more monitors. The one or more monitors may comprise an internal monitor positioned to face an operator of medical equipment, an external monitor position to face medical personnel using the medical equipment, or any combination thereof. The operator may comprise a physician, a surgeon, an attending physician, a resident, a nurse, a nurse practitioner, a surgical attendant, or any combination thereof. In some cases, the internal monitor and the external monitor may comprise different user interfaces, described elsewhere herein. In some instances, the one or more monitors may comprise at least two external monitors positioned to face medical personnel using the medical equipment, where the at least two external monitors comprise different user interfaces.

In some cases, the disclosure describes a method, comprising: displaying an object relative to a feature of an intravascular dataset or a feature of an extravascular dataset, where the feature of the intravascular dataset and the feature of the extravascular dataset are registered to a real-time extravascular dataset, and where the object is superimposed on a display of the real-time extravascular data.

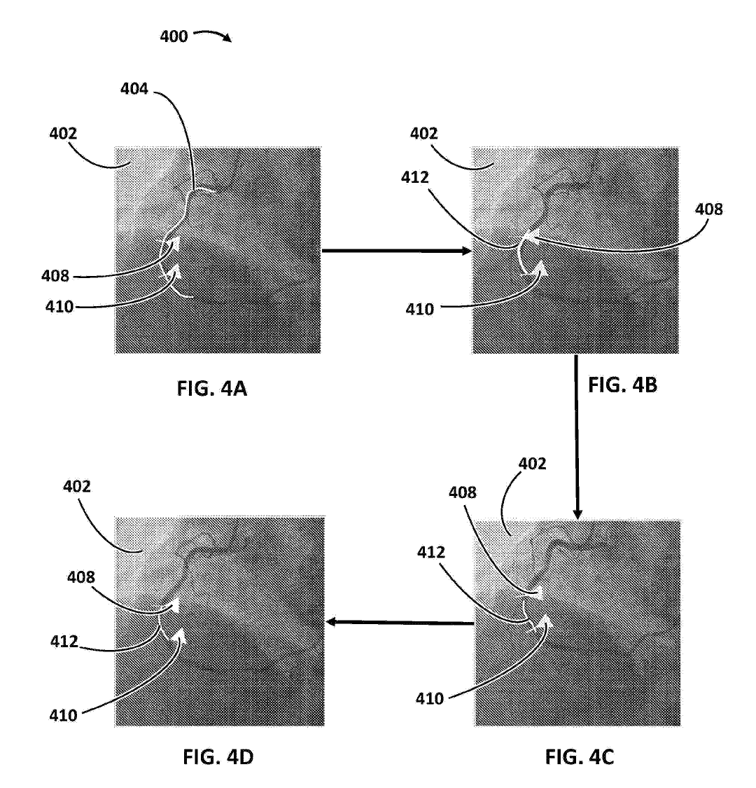

In some instances, the disclosure herein describes a method 400 of tracking a position of an intravascular device 412 with respect to the registered intravascular (404, 408, 410) and/or extravascular datasets 402, as seen in FIGS. 4A-4D. In some cases, the method may comprise: acquiring intravascular 404 and extravascular data 402; registering the intravascular and extravascular data; guiding a medical device 412 to an object (408, 410) registered to the intravascular data; and providing a visual indicator when the medical device is adjacent to the object (FIG. 4D). In some instances, the object comprises a first object 410 and a second object 408. In some cases, the visual indicator may be provided when the medical device is positioned between the first object 410 and the second object 408. In some cases, the indicator may comprise a color indicator, as shown in FIG. 4D. For example, a green color visual indicator may be provided when the medical device 412 is within a spatial positioned defined by the first object 410 and a second object 408. In some instances, the indicator may comprise a red color visual indicator that may be provided when the medical device 412 is outside of an area defined by the first object 410 and the second object 408. In some cases, the first object 410 and second object 408 may be set by a user or automatically by the system, as described elsewhere herein. In some cases, the medical device may comprise a catheter, as described elsewhere herein, and/or a catheter delivering a medical device (e.g., a stent).

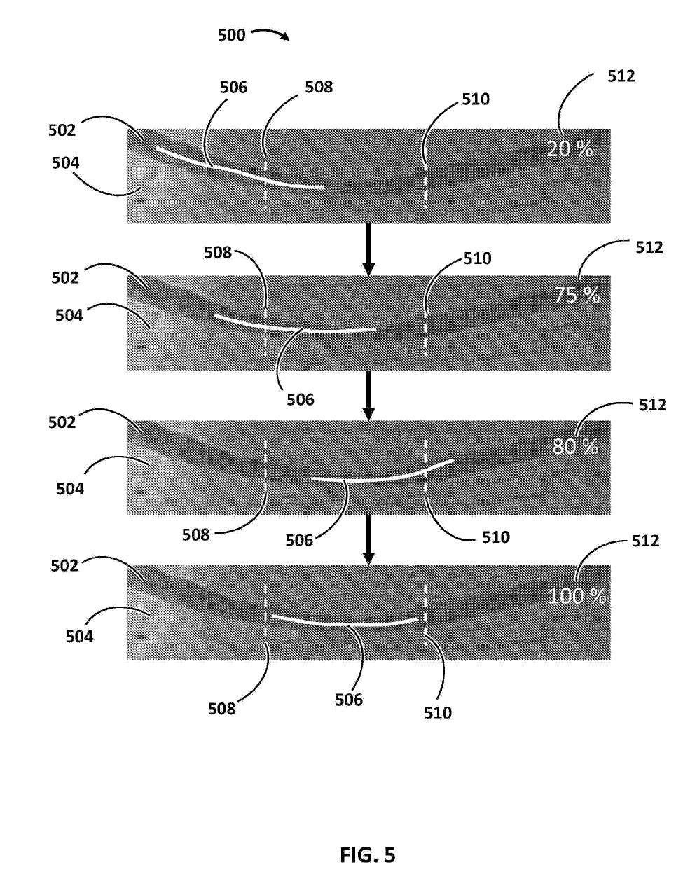

In some cases, an indicator may comprise a percentage indicator 512 of the position of the medical device 506 with respect to the first object 508 and/or the second object 510, as seen in a flow diagram 500 of FIG. 5. For example, the percentage indicator 512 may describe the percentage of the medical device within a spatial position range between the first object 508 and the second object 510. In some cases, a display of a cross-section of the blood vessel 502 from the extravascular data 504 may be superimposed with the medical device intravascular tracking position 506, the first object 508, and the second object 510. In some instances, the registered first object 508 and second object 510 may be registered to a real-time display of extravascular data and facilitate the real-time guidance and/or placement of the medical device within the blood vessel.

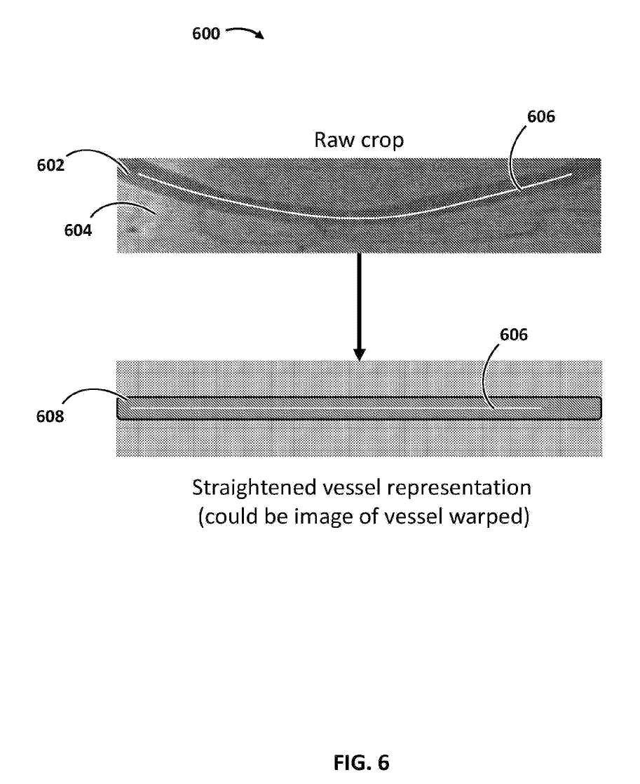

In some cases, the disclosure provided herein describes a method of processing registered intravascular and extravascular data 600, as seen in FIG. 6. In some instances, the method may comprise: obtaining intravascular 606 and/or extravascular data 604 of a vascular structure 602; determining the non-linear structure of the blood vessel from the intravascular data and/or the extravascular data; and processing the non-linear structure of the blood vessel to a linear structure 608. In some cases, processing the non-linear structure of the blood vessel to a linear structure may provide one or more features, e.g., determined by the one or more predictive models and/or machine learning algorithms that increase the accuracy of characterizing various tissue types and/or categories, described elsewhere herein. In some cases, the accuracy may increase by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 96%, at least about 97%, at least about 98%, or at least about 99%.

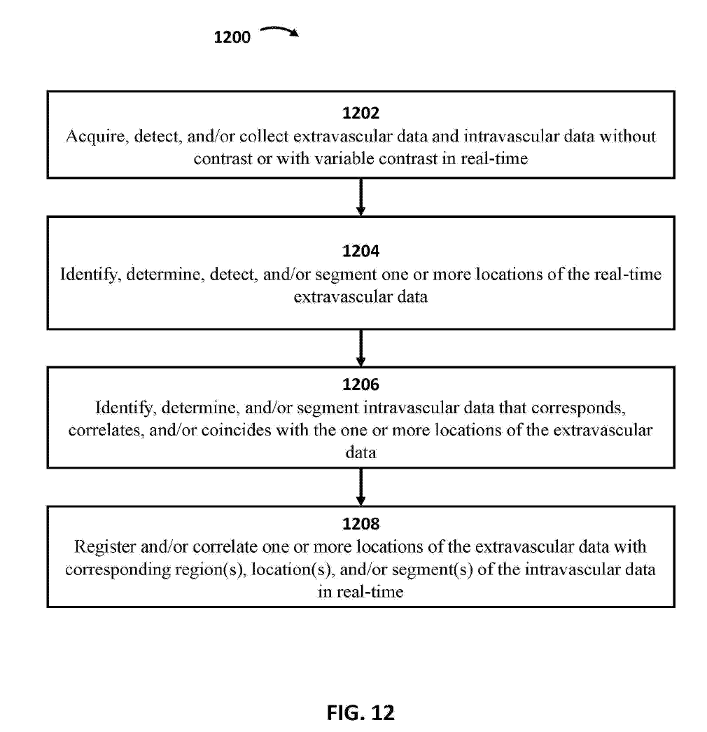

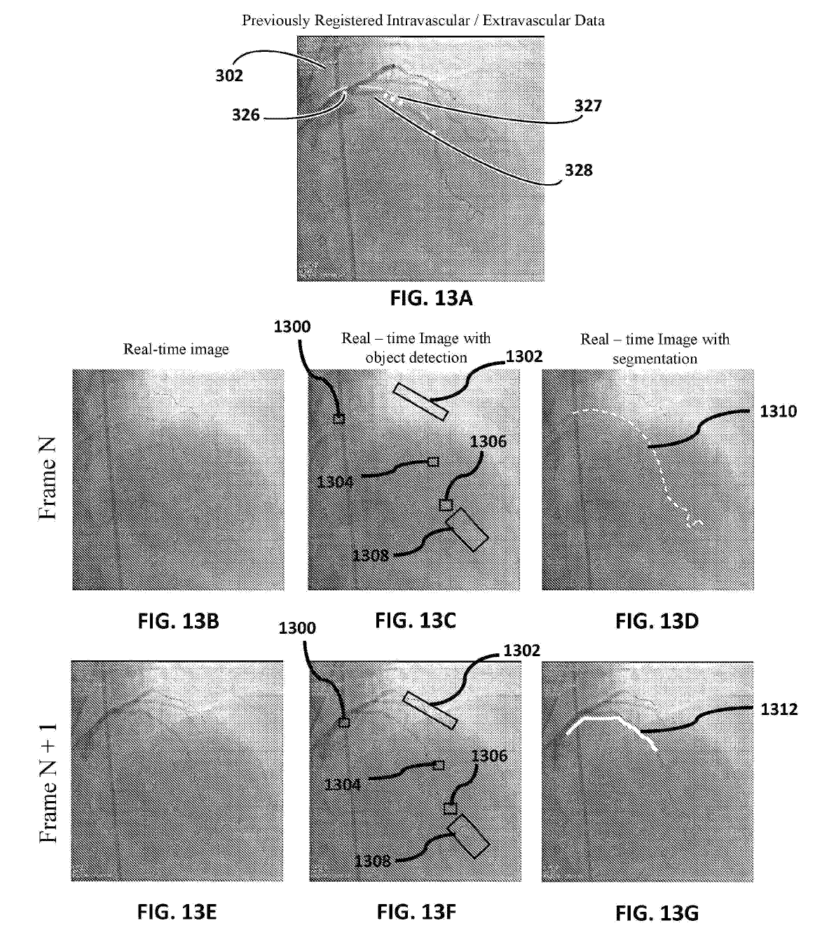

In some cases, the methods and/or systems for correlating and/or registering intravascular data and extravascular data in real-time may be operated and/or conducted without a contrast agent or with variable use of a contrast agent, as described elsewhere herein. In some cases, variable use of a contrast agent (as shown in FIGS. 13E, 13F, and 13G) may comprise providing and/or injecting contrast for less than about 1 second or less than about 2 seconds (i.e., a puff of contrast). In some cases, variable contrast may comprise providing and/or injecting contrast for up to about 2 instances of providing and/or injecting contrast to a subject’s vascular network during real-time extravascular and/or intravascular data collection. In some cases, the variable use of contrast agent may provide real-time updates of the registration and/or correlation of previously registered intravascular data and extravascular data, as shown in FIG. 13A. In some cases, the method for correlating and/or registering intravascular data and extravascular data without a contrast agent or with variable use of a contrast agent 1200, as shown in FIG. 12, may comprise: acquiring, detecting, and/or collecting extravascular data and intravascular data without contrast or with variable contrast in real-time 1202; identifying, determining, detecting, and/or segmenting one or more locations of the real-time extravascular data (1300, 1302, 1304, 1308, 1310 as shown in FIGS. 13B-C, 13D, and 13F) 1204; identifying, determining, detecting, and/or segmenting intravascular data (e.g., intravascular probe image data generated during a probe pull back) that corresponds, correlates, and/or coincides with the one or more locations within the extravascular data 1206; and registering and/or correlating the one or more locations of the extravascular data and the intravascular data in real-time 1208. In some cases, the one or more locations of the real-time extravascular data may be identified, determined, detected, and/or segmented with the use of a contrast agent, without the use of contrast agent, or with less than about 1 second of contrast or less than about 2 seconds of contrast provided and/or injected to a subject’s vascular network during real-time extravascular data and/or intravascular data collection. In some cases, the contrast may be provided and/or injected to a subject’s vascular network for up to about 2 instances of providing and/or injecting contrast to a subject’s vascular network during real-time extravascular data and/or intravascular data collection. In some cases, the identifying, determining, and/or segmenting of intravascular data may be conducted with no contrast provided and/or injected to a subject’s vascular network during real-time intravascular data collection. The one or more locations of the extravascular dataset may comprise a location and/or spatial position of a guide catheter tip 1300, primary guide wire tip 1306, secondary guide wire tip 1302, imaging catheter position marker 1304, distal imaging catheter marker 1306, guide wire path, guide wire position, any other location of extravascular data described elsewhere herein, or any combination thereof. In some cases, the imaging catheter position marker may comprise a radio-opaque catheter marker visualized and/or detected as shown in extravascular data. In some cases, the variable use of the contrast agent may segment one or more vessels’ geometry 1312, as shown in FIG. 13G. In some instances, the identifying, determining, detecting, collecting, and/or segmenting of one or more locations of the extravascular data may be conducted automatically e.g., by one or more processors of the systems, described elsewhere herein. In some cases, the methods and/or systems to guide, provide, and/or implant an object (e.g., a stent) to one or more locations within the extravascular data and/or the intravascular data, described elsewhere herein, may be conducted and/or operated with the use of contrast or without the use of contrast.

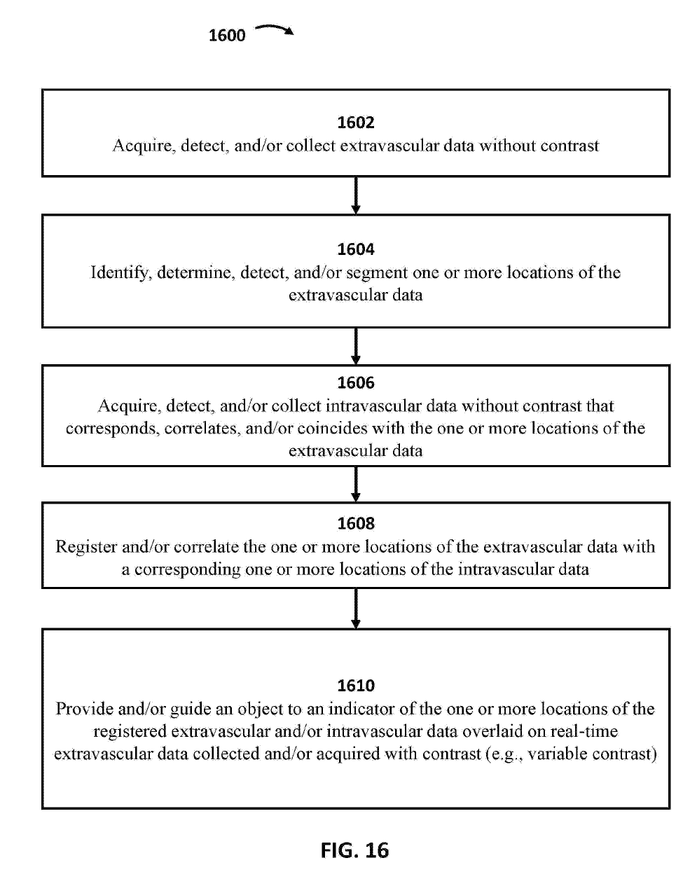

In some cases, the method 1600 may comprise collecting, acquiring, and/or detecting intravascular data and extravascular data without injecting and/or providing contrast to a subject’s vascular network while providing and/or injecting contrast when guiding, implanting, and/or providing an object to one or more locations with the extravascular data and/or the intravascular data with contrast, as shown in FIG. 16. In some cases, the method 1600, may comprise: acquiring, detecting, and/or collecting extravascular data without contrast 1602; identifying, determining, detecting, and/or segmenting one or more locations of the real-time extravascular data 1604; acquiring, detecting, and/or collecting intravascular data without contrast that corresponds, correlates, and/or coincides with the one or more locations of the extravascular data 1606; registering and/or correlating the one or more locations of the extravascular data with the corresponding one or more locations of the intravascular data 1608; and providing and/or guiding an object to an indicator of the one or more locations of the registered extravascular and/or intravascular data overlaid on real-time extravascular data collected and/or acquired with contrast (e.g., variable contrast) 1610. In some cases, the real-time extravascular data collected and/or acquired with contrast may be registered to one or more locations of the registered extravascular and intravascular data collected and/or acquired without contrast. In some cases, acquiring, detecting and/or collecting intravascular and extravascular data may occur simultaneously. In some cases, the intravascular and/or extravascular data acquired without contrast may be acquired in real-time, as described elsewhere herein. In some cases, the one or more locations of the intravascular data and/or the extravascular data may comprise one or more regions, one or more segments, or a combination thereof the intravascular data and/or the extravascular data. In some cases, the intravascular data may be collected and/or detected with an intravascular imaging probe, as described elsewhere herein. In some cases, the object may comprise a stent, as described elsewhere herein. In some instances, contrast provided and/or injected to a subject’s vascular network may comprise injecting and/or providing contrast for less than about 1 second, or less than about 2 seconds. In some instances, the contrast may be provided for up to about 2 instances of providing and/or injecting contrast to a subject’s vascular network during real-time intravascular data and/or extravascular data collection.

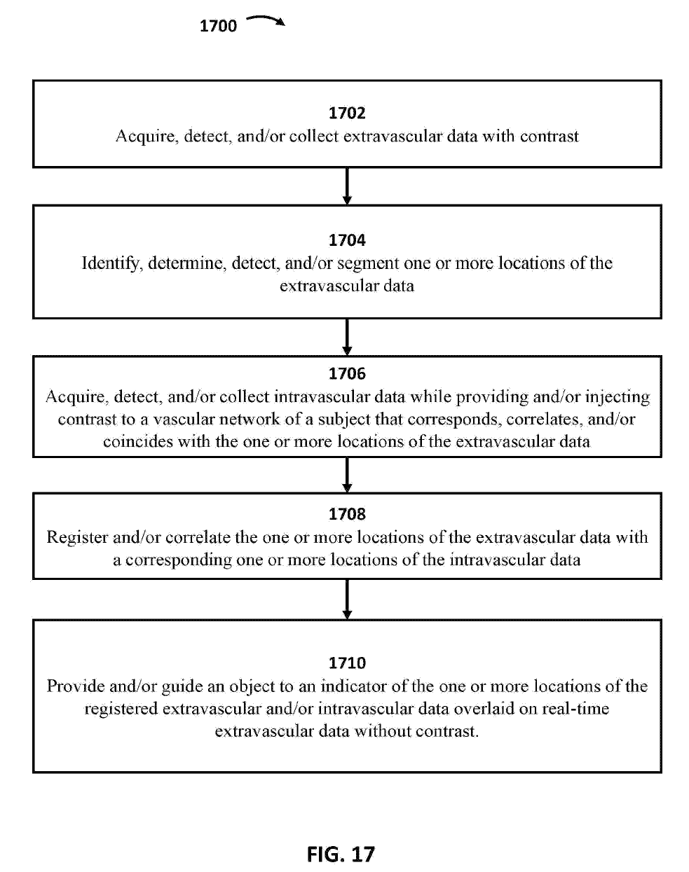

In some cases, the method 1700 may comprise collecting, acquiring, and/or detecting intravascular data and extravascular data while injecting and/or providing contrast to a subject’s vascular network and then collecting, acquiring, and/or detecting real-time, contrast-free extravascular data while guiding, implanting, and/or providing an object to one or more locations of the extravascular data and/or the intravascular data, as shown in FIG. 17. In some cases, the method 1700, may comprise: acquiring, detecting, and/or collecting extravascular data with contrast 1702; identifying, determining, detecting, and/or segmenting one or more locations of the extravascular data 1704; acquire, detect and/or collect intravascular data while providing and/or injecting contrast to a vascular network of a subject that corresponds, correlates, and/or coincides with the one or more locations extravascular data 1706; registering and/or correlating the one or more locations of the extravascular data with a corresponding one or more locations of the intravascular data 1708; and providing and/or guiding an object to an indicator of the one or more locations of the registered extravascular and/or intravascular data overlaid on real-time extravascular data collected and/or acquired without contrast 1710. In some cases, the real-time extravascular data collected and/or acquired without contrast may be registered to one or more locations of the registered extravascular and intravascular data collected and/or acquired with contrast. In some cases, acquiring, detecting and/or collecting intravascular and extravascular data may occur simultaneously. In some cases, the intravascular and/or extravascular data acquired with contrast may be acquired in real-time, as described elsewhere herein. In some cases, the one or more locations of the intravascular data and/or the extravascular data may comprise one or more regions, one or more segments, or a combination thereof the intravascular data and/or the extravascular data. In some cases, the intravascular data may be collected and/or detected with an intravascular imaging probe, as described elsewhere herein. In some cases, the object may comprise a stent, as described elsewhere herein. In some instances, contrast provided and/or injected to a subject’s vascular network may comprise injecting and/or providing contrast for less than about 1 second, or less than about 2 seconds. In some instances, the contrast may be provided for up to about 2 instances of providing and/or injecting contrast to a subject’s vascular network during real-time intravascular data and/or extravascular data collection.

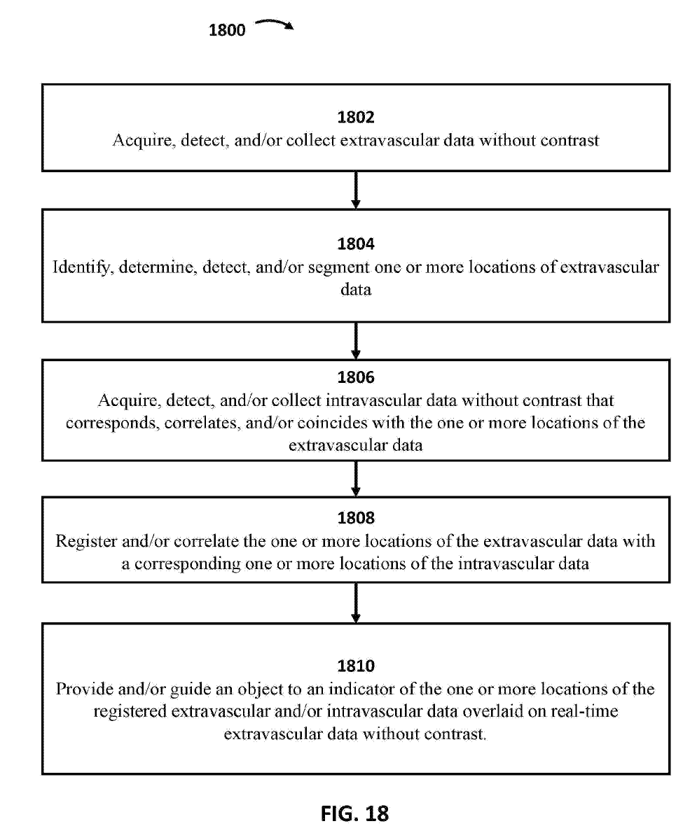

In some cases, the method 1800 may comprise collecting, acquiring, and/or detecting intravascular data and/or extravascular data without contrast and guiding, implanting, and/or providing an object to one or more locations of the extravascular data and/or the intravascular data without contrast, as shown in FIG. 18. In some cases, the method 1800, may comprise: acquiring, detecting, and/or collecting extravascular data without contrast 1802; identifying, determining, detecting, and/or segmenting one or more locations of the extravascular data 1804; acquiring, detecting, and/or collecting intravascular data without contrast that corresponds, correlates, and/or coincides with the one or more locations of the extravascular data 1806; registering and/or correlating the one or more locations of the extravascular data with a corresponding one or more locations of the intravascular data 1808; and providing and/or guiding an object to an indicator of the one or more locations of the registered extravascular and/or intravascular data overlaid on real-time extravascular data without contrast 1810. In some cases, the real-time extravascular data without contrast may be registered and/or correlated to the registered extravascular and intravascular data acquired without contrast. In some cases, acquiring, detecting and/or collecting intravascular and extravascular data may occur simultaneously. In some cases, the intravascular and/or extravascular data acquired without contrast may be acquired in real-time, as described elsewhere herein. In some cases, the one or more locations of the intravascular data and/or the extravascular data may comprise one or more regions, one or more segments, or a combination thereof the intravascular data and/or the extravascular data. In some cases, the intravascular data may be collected and/or detected with an intravascular imaging probe, as described elsewhere herein. In some cases, the object may comprise a stent, as described elsewhere herein.

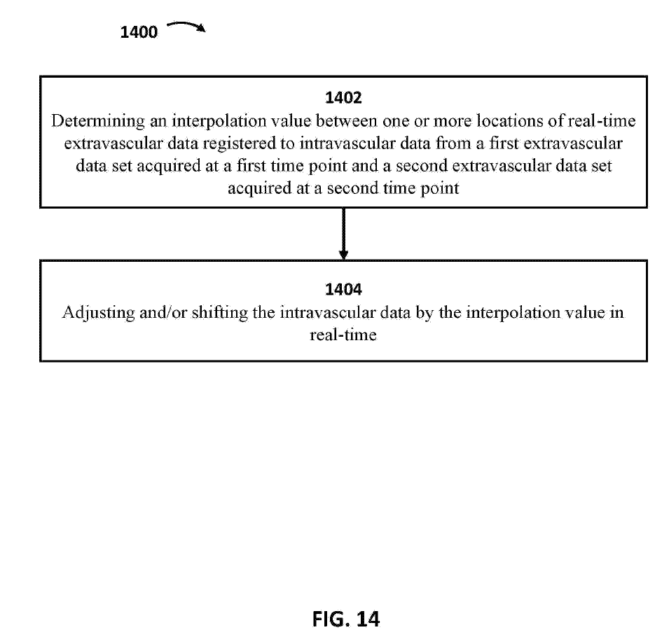

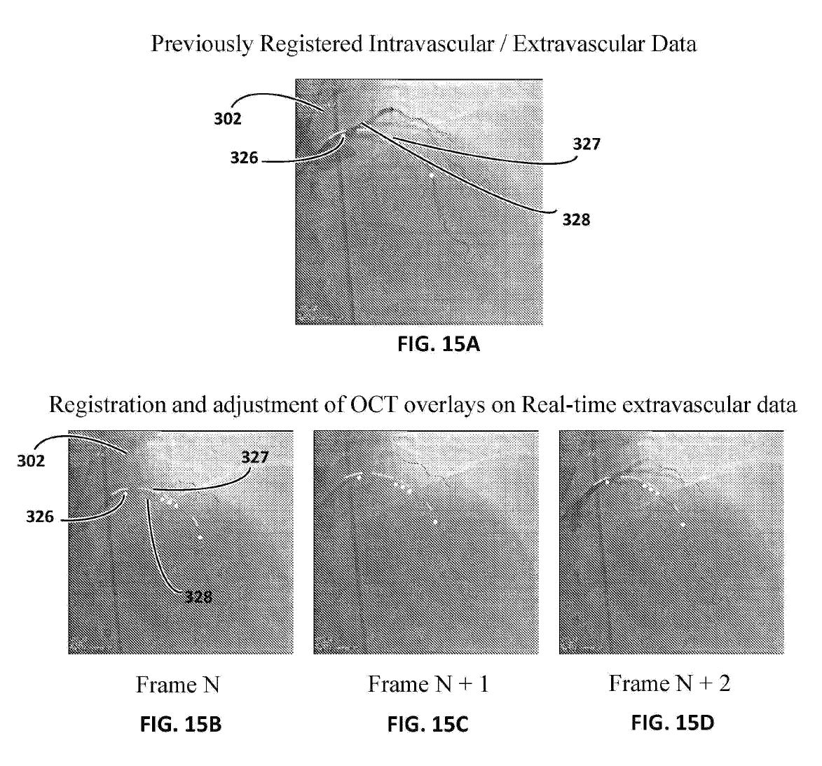

In some cases, a previously registered position of intravascular data registered to extravascular data, as shown in FIG. 15A, may shift or change in location and/or spatial position over time (e.g., during real-time extravascular or intravascular data collection) as one or more locations of the extravascular data spatially move as a result of a subject’s beating heart, inhaling and exhaling, or movement of the subject. In some cases, the methods 1400 (e.g., as shown in FIG. 14), implemented with the systems described elsewhere herein, may correct for and/or adjust the position and/or location of the registered intravascular data in real-time, as shown in FIGS. 15B-15D. In some cases, the methods and system may adjust the position of the registered intravascular data in real-time by: determining an interpolation value between the one or more locations of the extravascular data registered to intravascular data, from a first extravascular data set acquired at a first time point and a second extravascular data set acquired at a second time point, where the first time point precedes the second time point (e.g., between successive and/or subsequent extravascular data acquired in real-time) 1402; and adjusting and/or shifting a position and/or location of the intravascular data by the interpolation value 1404. In some cases, the adjusting and/or shifting of the position and/or location of intravascular data by the interpolation value may be conducted in real-time. In some cases, the interpolation value may comprise a translation of one or more locations of the extravascular data in a at least 1-dimension, at least 2 dimensions, or at least 3 dimensions. In some cases, the translation value may comprise a value of shifting a position of the intravascular data by a first dimension and a second dimension. In some instances, the first dimension, second dimension, and third dimension may be orthogonal to one another. In some cases, a first extravascular dataset (e.g., an extravascular image) acquired at a first time point may comprise a first interpolation value and wherein a second extravascular dataset acquired at a later second time point after the first time point in time may comprise a different interpolation value. In some cases, the interpolation value may comprise a distance of a shift for the one or more locations of the extravascular data. In some cases, the one or more locations of the extravascular data may comprise a location and/or spatial position of a guide catheter tip 1300, primary guide wire tip 1306, secondary guide wire tip 1302, imaging catheter position marker 1304, distal imaging catheter marker 1306, guide wire path, guide wire position, any other location of extravascular data described elsewhere herein, or any combination thereof. In some cases, the one or more locations of the extravascular data may be determined automatically, e.g., through the use of one or more algorithms or predictive models, as described elsewhere herein. In some cases, the one or more locations of the extravascular data may be determined in real-time.

### User Interfaces

Aspects of the systems of disclosure provided herein may comprise one or more view configurations (i.e., interface(s) (UI)) 1116 displaying on one or more data views 300 comprising e.g., an extravascular data view (302), an intravascular data view (320, 304), as shown in FIGS. 3A-3C, on one or more monitors (102, 104), as shown in FIG. 1A. The UI 1116 may be displayed on a flat-screen panel or a touch-screen display 1114. In some embodiments, the user-interface 1116 may comprise a touch screen interface permitting a user to tap on the screen to select operations and/or to interact with data. In some instances, the user-interface 1116 may be manipulated or interacted with a keyboard and/or mouse. In some instances, the user-interface 1116 may be manipulated using a separate piece of hardware located near the end-user and the patient.

The UI 1116 may display intravascular data, extravascular data, registered intravascular and extravascular data, or any combination thereof. The user-interface may provide actionable information for health care personnel to guide diagnosis of coronary artery disease. In some cases, the user-interface may comprise a data view indicating measured parameters of distance between an imaging probe and an object superimposed onto a registered intravascular and extravascular image.

In some embodiments, the user-interface 1116 may comprise one or more buttons (330, 306, 318), switches, editable dialogue boxes, sliders, radio buttons, or any combination thereof. In some instances, the user-interface may comprise menus that allow the user to configure device parameters e.g., scanning speed, resolution, or any combination thereof imaging system parameters. The user-interface may comprise functional buttons that may toggle between varying views of extravascular data with or without overlay of an object and/or of registered intravascular data. In some cases, the user-interface may comprise functional buttons that enable scanning, stop scanning, emergency stop scanning, pause scanning, resume scanning, or any combination thereof.

In some cases, the intravascular (320, 304) views may comprise a sagittal and/or circular cross-section view 320, and a longitudinal vessel cross-section 304. The circular cross-section view 320 may display circular cross-section intravascular data and one or more annotations e.g., user and/or machine determine segments of tissue region of interest 321, center of the imaging probe, overlayed biochemical signature indicators 332, or any combination thereof. In some instances, the circular cross-section view 320 may also display a measurement tool 322 configured to generate one or more measured parameters. In some cases, when a user interacts (e.g., clicking, touch, and/or pressing) the measurement tool 322, the cursor or pointer may change to a measurement cursor or pointer where the user may interact with the circular cross- section view 320 to measure one or more parameters 319, described elsewhere herein. In some cases, when a user interacts (e.g., clicking, touching, and/or pressing) the measurement tool 322 the systems and one or more algorithms, described elsewhere herein, may generate one or more measured parameters 319 automatically. The circular cross-section view 320 may display one or more measured parameters 319 of the blood vessel e.g., external elastic membrane (EEM) 321, lumen, stenosis, lumen area, or any combination thereof.

The longitudinal vessel cross-section view 304 may display a contour of the inner lumen of a blood vessel 308 as a function of the length of the blood vessel. In some cases, the longitudinal vessel cross-section view may display a scale 323 of the intravascular data displayed in the data view. The longitudinal vessel cross-section view 304 may comprise corresponding indicators of regions along the blood vessel length where one or more biochemical spectroscopy signals (310, 314), e.g., calcium, lipid, etc., have been detected. In some instances, a user may interact with the longitudinal vessel cross-section view 304 by scrubbing or scrolling through the length of the longitudinal cross-sectional view with a cursor 315 to display a corresponding circular cross-sectional image at the cursor position of the longitudinal cross-sectional view in the circular or cross-sectional view 320.

In some cases, one or more objects (312, 316, 325, 309), described elsewhere herein, may be added and/or overlaid on the longitudinal vessel cross-section view (304), as seen in FIGS. 3A-3C. In some instances, the one or more objects (312, 316, 325, 309) may be added and/or overlaid by a user and/or by a predictive model (e.g., a machine learning model and/or algorithm). The one or more objects may comprise a first object 312, a second object 316, a third object 325, a fourth object 309, or any combination thereof objects, described elsewhere herein. The user may place the one or more objects (312, 316, 325, 309) by clicking or tapping on a region of the displayed imaged contour of the blood vessel 308 to display an object set dialogue. By tapping and/or clicking the one or more buttons of the view and/or view configuration, the user may then confirm the placement of one or more objects (312, 316, 325, 309). Annotations and/or markup of the circular cross-sectional view 320 may be displayed on the longitudinal vessel cross-section view 304. For example, the line annotation of the cross-sectional view 321, as shown in FIGS. 3B-3C, may be displayed as a similar line annotation 309 in the longitudinal vessel cross-section view 304. In some instances, the fourth object 309 may comprise an indicator of a position and/or region of the external elastic membrane of a blood vessel.

In some cases, the first object 312 may comprise a first visual indicator 328 and the second object 316 may comprise a second visual indicator 329. In some instances, the first visual indicator 328 and/or the second visual indicator 329 may display one or more measured parameter. The one or more measured parameters may comprise a diameter of the external elastic membrane, lumen diameter, lumen area, or any combination thereof. In some instances, the one or more measured parameters may be measured by a user and/or by one or more algorithms and/or predictive models of the system described elsewhere herein. In some instances, a minimum lumen area 331 may be determined and/or displayed along the longitudinal vessel cross-section view 304. In some cases, the minimum lumen area 331 may be determined and/or set by a user, one or more algorithms, and/or one or more predictive models of the system, described elsewhere herein. In some cases, a distance between the first object 312 and the second object 316 may be determined by a user and/or the system (i.e., one or more algorithms and/or predictive models, described elsewhere herein) and displayed on the longitudinal vessel cross-section view 304. In some cases, the third object 325 may comprise a marker or indicator (e.g., a square or a circle) superimposed on longitudinal vessel cross-section view 304. In some instances, the marker or indicator 325 may indicate a branch of a blood vessel.

In some cases, the extravascular data view 302 may display an overlay of the registered one or more objects (324, 326, 327, 328, 333), as seen in FIGS. 3B-3C. In some cases, the registered one or more objects (324, 326, 327, 328, 333) may be set by a user and/or predictive model from the intravascular data and/or corresponding intravascular data view (304, 320). In some instances, the registered one or more objects may comprise a scan path traversed by the imaging probe, described elsewhere herein, that may be displayed in a corresponding registered location of a blood vessel in the extravascular data view 302. In some instances, the registered one or more objects may be registered to the extravascular data and displayed in the extravascular data view 302 that track movements of a subject as real-time extravascular data is displayed in the extravascular data view 302. In some cases, the registered one or more objects (327, 328) may comprise objects corresponding to the location of the one or more biochemical spectroscopy signals (310, 314), e.g., calcium, lipid, etc., have been detected as shown in the longitudinal vessel cross-section view 304. In some cases, the registered one or more objects (327, 328, 314, 310) may comprise differing color indicators e.g., yellow corresponding to lipid and blue corresponding to calcium as shown in FIGS. 3A-3C. In some instances, an object 326 of the registered one or more objects may comprise a corresponding marker or indicator 326, that corresponds and/or is correlated to the marker or indicator 325 displayed on the longitudinal vessel cross-section view 304, described elsewhere herein. In some cases, the registered one or more objects may comprise an object 333 that corresponds and/or is registered to a location and/or position of the first object 312 as displayed in the longitudinal vessel cross-section view 304. In some instances, the registered one or more objects may comprise an object 324 that corresponds and/or is registered to a location and/or position of the second object 316 as displayed in the longitudinal vessel cross-section view 304.

In some cases, the user interface may comprise one or more view configurations (i.e., user interfaces), as shown in FIGS. 7A-10. In some cases, the one or more view configurations (700, 716, 724) may comprise one or more data views (710, 712, 714, 726) e.g., extravascular data view and/or intravascular data views with one or more user interface elements (702, 704, 706, 708) (e.g., a button or tab) that when activated switch between e.g., first view configuration 700, second view configuration 716, or third view configuration 724. In some cases, the one or more user interface elements may correspond to one or more view configurations of one or more subjects and/or patients imaged with the systems and methods, described elsewhere herein. In some instances, the one or more view configurations may comprise the same number, arrangement, size, or any combination thereof characteristics of data views, as seen in FIGS. 7A-7B. In some cases, the one or more view configurations may comprise differing number, arrangement, size, or any combination thereof characteristics of data views 726, as seen in FIG. 7C, compared to the data views (710, 712, 714) shown in FIG. 7A.

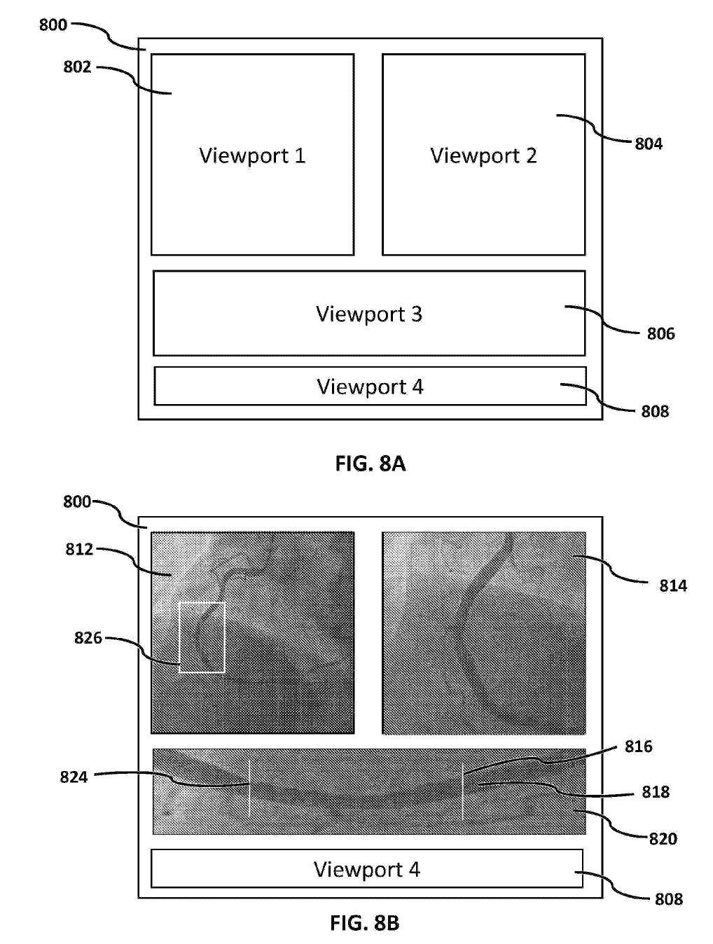

In some instances, the user interface may comprise one or more data views that may display data e.g., extravascular data, intravascular data, or a combination thereof data, as described elsewhere herein. In some cases, the user interface 800 may comprise one or more data views (802, 804, 806, 808) that display data of a single type (812, 814, 820), as shown in FIGS. 8A-8B. In some instances, the one or more data views may display data of a single type, e.g., extravascular data from one or more imaging perspectives. For example, a first data view (802, 812) of the one or more data views may display macroscopic extravascular data collected by e.g., x-ray angiography. A second data view (804, 814) of the one or more data views may display a zoomed in portion of the first data view (802, 812). In some cases, a user and/or a predictive model and/or machine learning model implemented by the systems, described elsewhere herein, may select a region of interest 826 of a first data view (802, 812) that may be displayed in the second data view (804, 814). In some instances, the region of interest 826 may be dynamically adjusted and/or moved across the first data view (802, 812) and the corresponding display of the second data view (804, 814) may updated accordingly based on the adjusted region of interest 826. In some cases, the one or more data views may comprise a third data view (820, 806). The third data view (820, 806) may display a cross-sectional imaging perspective of the first and/or second data view (802, 812; and 804, 814). In some instances, the region of interest 826 of the first data view (802, 812) may be displayed in the third data view (824, 816). In some cases, adjustment of the region of interest 826 in the first data view (802, 812) may adjust the corresponding data displayed in the third data view (824, 816) according to the adjusted area covered by the region of interest. In some cases, a fourth data view 808 may display intravascular data, extravascular data, measurements, or any combination thereof, as described elsewhere herein, in the form of a graphical representation (e.g., to reduce display overlays on the other data views).

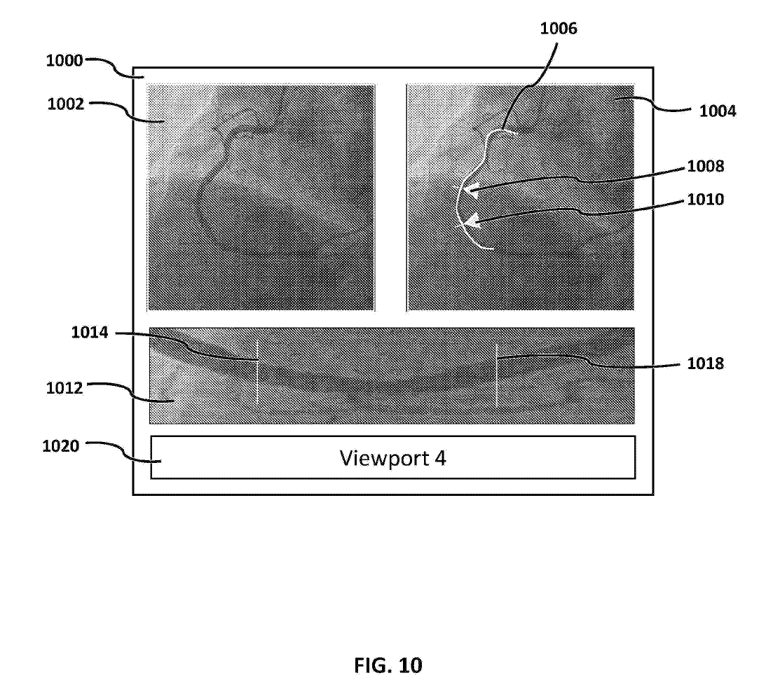

In some instances, for example, a second data view 1004 of one or more data views (1002, 1104, 1012, 1020) may comprise one or more objects and/or annotations of registered intravascular and extravascular data (1006, 1108, 1010), as seen in the view configuration 1000 of FIG. 10. In some cases, the one or more objects and/or annotations of the registered intravascular and extravascular data may comprise the path of an imaging probe and/or medical device 1006, a first object 1008, a second object 1010, or any combination thereof. In some cases, a third data view 1012 of extravascular and/or intravascular data may comprise one or more objects and/or annotations (1014, 1018) corresponding to the one or more objects and/or annotations (1006, 1008, 1010) of the second data view 1004. In some cases, the one or more objects and/or annotations of the second data view 1004 may be adjusted by a user and/or a predictive model and/or algorithm, described elsewhere herein, and the corresponding adjusted one or more objects and/or annotations displayed in the second data view may update in a corresponding manner as shown in the third data view 1012. In some cases, the one or more objects and/or annotations (1014, 1018) displayed in the third data view may be adjusted and/or moved by a user and/or predictive model and/or algorithm (e.g., machine learning algorithm), where after adjusting the one or more objects and/or annotations a corresponding location of the one or more objects and/or annotations (1006, 1008, 1010) may adjust as displayed in the second data view 1004. In some cases, a fourth data view 1020 may display intravascular data, extravascular data, measurements, or any combination thereof, as described elsewhere herein, in the form of a graphical representation (e.g., to reduce display overlays on the other data views).

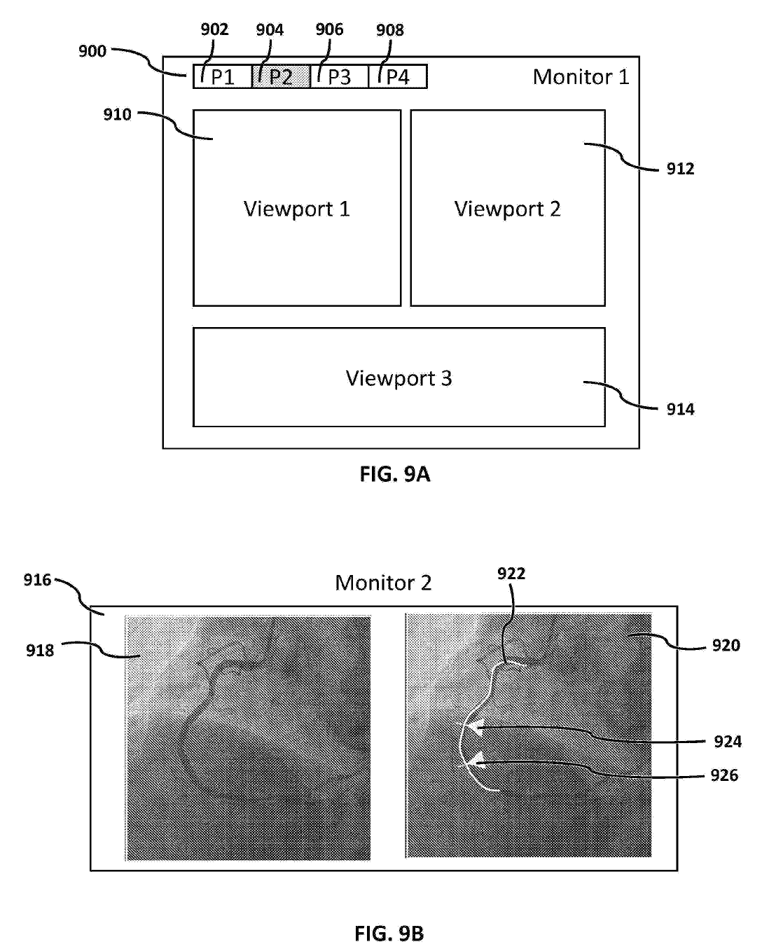

In some cases, the one or more monitors of the system, described elsewhere herein may display one or more view configurations (900, 916), as seen in FIGS. 9A-9B. In some cases, the one or more monitors may comprise a first monitor 900 with a first view configuration comprised of one or more data views (910, 912, 914) and a second monitor 916 with a second view configuration comprised of one or more data views (918, 920). In some cases, the first view configuration and the second view configuration may be the same. In some instances, the first view configuration and the second view configuration may differ. In some instances, the first data view configuration may comprise one or more user interface elements (902, 904, 906, 908), described elsewhere herein, where the second data view configuration 916 does not. In some instances, the first monitor and the first view configuration may be utilized by a device operator, nurse, scrub technician, and/or other medical personnel not conducting the medical procedure i.e., not the attending physician and/or medical doctor guiding the imaging probe and/or medical device through a blood vessel as described elsewhere herein. In some cases, the second monitor display may be a guide for the attending physician and/or medical doctor when conducting the imaging procedure. In some instances, the second view configuration 916 may comprise one or more views of extravascular data, intravascular data, or a combination thereof data. In some cases, the one or more data views (918, 920) may comprise data of the same type. In some instances, the one or more data views may comprise data of differing types. In some cases, the one or more data views may display data with registered intravascular (922, 924, 926) and extravascular data 920, as shown in FIG. 9B. In some cases, the registered intravascular data (922, 924, 926) may comprise the scan path of the imaging probe 922 and/or one or more objects (924, 926), described elsewhere herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,”, “patient”, or “subject” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject’s body. The term “ex vivo” is used to describe an event that takes place outside of a subject’s body. An ex vivo assay is not performed on a subject. Rather, it is performed upon a sample separate from a subject. An example of an ex vivo assay performed on a sample is an “in vitro” assay. The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

Any systems, methods, software, compositions, and platforms described herein are modular and not limited to sequential steps. Accordingly, terms such as “first” and “second” do not necessarily imply priority, order of importance, or order of acts.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### Examples

### Example 1: Registration between contrast free extravascular data and intravascular data with variable use of contrast during cardiovascular stent placement

The methods and systems of the disclosure, described elsewhere herein, are capable of registering extravascular data and intravascular data without the use of or with variable use of contrast when acquiring real-time extravascular and intravascular data. The variable use of contrast involves providing and/or injecting contrast for less than about 1 second or less than about 2 seconds (i.e., a puff of contrast) for up to about 2 instances of providing and/or injecting contrast to a subject’s vascular network during real-time extravascular data collection, as described elsewhere herein.

Injecting and/or providing variable amount contrast can be used during registration of extravascular and intravascular data during a cardiovascular stenting procedure for a subject. An example of such a procedure includes the steps of: acquiring, collecting, and/or detecting real-time extravascular data (e.g., angiography fluoroscopy data) of one or more locations (e.g., the location of non-contrasted objects such as a guide wire tip, described elsewhere herein) of the extravascular data without contrast; acquiring, collecting, and/or detecting real-time intravascular data (e.g., intravascular OCT and/or near infrared spectroscopy data) by rotating and translating a combined intravascular OCT and near infrared spectroscopy probe, where the position and/or location of the probe is determined and/or detected in the extravascular data without the use of contrast and based on at least an imaging marker of the probe visualized in the extravascular data; registering and/or correlating one or more locations of the extravascular data with a corresponding one or more locations of the intravascular data; and guiding and placing an object (e.g., a cardiovascular stent) to/at an indicator of the one or more locations of the registered intravascular and/or extravascular data overlaid on real-time extravascular data collected and/or acquired when providing or injecting a variable amount of contrast agent into the subject’s vascular network to visualize the vasculature morphology and placement of the stent. Such an example use of variable contrast reduces the overall use of contrast and the potential side effects of the use of typical amounts of contrast for subjects undergoing a guided angiography with fluoroscopy.

### Example 2: Registration between contrast-filled extravascular data and intravascular data with no use of contrast during cardiovascular stent placement

The methods and/or systems of the disclosure, described elsewhere herein, are capable of registering extravascular data and intravascular data without the use of or with variable use of contrast when acquiring real-time extravascular. The variable use of contrast involves providing and/or injecting contrast for less than about 1 second or less than about 2 seconds (i.e., a puff of contrast) for up to about 2 instances of providing and/or injecting contrast to a subject’s vascular network during real-time extravascular data collection, as described elsewhere herein.

Injecting and/or providing variable amount contrast can be used during registration of extravascular and intravascular data during a cardiovascular stenting procedure for a subject. An example of such a procedure includes the steps of: acquiring, collecting, and/or detecting extravascular data (e.g., angiography fluoroscopy data) of one or more locations (e.g., the location of non-contrasted objects such as a guide wire tip, described elsewhere herein) of the extravascular data with contrast; acquiring, collecting, and/or detecting intravascular data (e.g., intravascular OCT and/or near infrared spectroscopy data) by rotating and translating a combined intravascular OCT and near infrared spectroscopy probe, where the position and/or location of the probe is determined and/or detected in the extravascular data with the use of contrast and is based on at least an imaging marker of the probe visualized in the extravascular data; registering and/or correlating one or more locations of the extravascular data with a corresponding one or more locations of the intravascular data; and guiding a procedure and/or placing an object (e.g., a cardiovascular stent) with real-time extravascular data without contrast, to/at an indicator of the one or more locations of registered extravascular and/or intravascular data that is overlaid on the real-time extravascular data. Such an example use of variable contrast during the real-time portion of the procedure reduces the overall use of contrast and the potential side effects of the use of typical amounts of contrast for subjects undergoing a guided angiography with fluoroscopy.

### Example 3: Registration between contrast-free extravascular and intravascular data with no use of contrast during cardiovascular stent placement

The methods and/or systems of the disclosure, described elsewhere herein, are capable of acquiring and/or registering extravascular data and intravascular data without the use of contrast. The extravascular data and the intravascular data may then be used to guide placement of an object (e.g., a cardiovascular stent), without the need of using contrast (e.g., during a real-time acquisition of extravascular data).

An example of such a procedure includes the steps of: acquiring, collecting, and/or detecting extravascular data (e.g., angiography fluoroscopy data) of one or more locations (e.g., the location of objects such as a guide wire tip, described elsewhere herein) of the extravascular data without contrast; acquiring, collecting, and/or detecting intravascular data (e.g., intravascular OCT and/or near infrared spectroscopy data) by rotating and translating a combined intravascular OCT and near infrared spectroscopy probe, where the position and/or location of the probe is determined and/or detected in the extravascular data without the use of contrast and based on at least an imaging marker of the probe visualized in the extravascular data; acquiring real-time extravascular data without contrast; registering and/or correlating one or more locations of the extravascular data to a corresponding one or more locations of the intravascular data collected and/or acquired without contrast; and guiding a procedure and/or placing an object (e.g., a cardiovascular stent) to/at an indicator of the one or more locations of the registered one or more locations of the extravascular and/or intravascular data overlaid on real-time extravascular data collected and/or acquired without contrast. Such an example use of no contrast exemplifies a method of the disclosure, provided elsewhere herein, by which no potential side effects from the use of contrast are exhibited by subjects undergoing a guided angiography with fluoroscopy.

Although the steps of the methods described elsewhere herein are described with a sequential order of the steps, as described elsewhere herein, a person of ordinary skill in the art will recognize many variations based on the teaching described herein. The steps may be completed in a different order. Steps may be added or omitted. Some of the steps may comprise sub-steps. Many of the steps may be repeated as often as beneficial. One or more of the steps of the methods, described elsewhere herein, may be acted upon or completed simultaneously. One or more of the steps of the methods may be performed with circuitry as described herein, for example, one or more of the processors or logic circuitry of a computer system or processing architecture.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A system, comprising:
an intravascular device configured to be positioned within a blood vessel of a patient; and
a processor configured for communication with the intravascular device and a display, wherein the processor is configured to:
use the intravascular catheter to acquire intravascular data of the blood vessel, wherein the intravascular data comprises biochemical data;
determine, from the biochemical data, where a biochemical composition is present within the blood vessel;
acquire a real-time stream of extravascular data of the blood vessel;
register the intravascular data with the extravascular data, thereby identifying a registered location within the extravascular data, wherein the registered location corresponds to where the biochemical composition is present; and
provide, on the display:
the real-time stream of the extravascular data; and
a biochemical composition indicator in the real-time stream of the extravascular data,
wherein a position of the biochemical composition indicator on the display corresponds to the registered location.

2. The system of claim 1, wherein the biochemical composition indicator is superimposed on the real-time stream of the extravascular data.

3. The system of claim 1, wherein the biochemical composition comprises a lipid or calcium.

4. The system of claim 1, wherein the biochemical composition indicator comprises a color that represents the biochemical composition.

5. The system of claim 4, wherein the color comprises a first color when the biochemical composition comprises a lipid and a different, second color when the biochemical composition comprises calcium.

6. The system of claim 1, wherein the biochemical data comprises near infrared spectroscopy (NIRS) data.

7. The system of claim 1,
further comprising a multi-modal imaging system comprising the intravascular device,
wherein the multi-modal imaging system comprises a combined optical coherence tomography (OCT) and near infrared spectroscopy (NIRS) imaging system.

8. The system of claim 1, wherein the processor is configured to determine, using a predictive model, where the biochemical composition is present within the blood vessel.

9. The system of claim 1, wherein the extravascular data comprises x-ray data.

10. The system of claim 1,
wherein the processor is further configured to provide, on the display, a longitudinal cross-section view of the blood vessel,
wherein the longitudinal cross-section view comprises an indicator of a region of the blood vessel where the biochemical composition is present.

11. The system of claim 1, wherein the biochemical composition indicator is switchable between a first state in which the biochemical composition indicator is visible and a second state in which the biochemical composition indicator is not visible.

12. The system of claim 1,
wherein the biochemical composition is a first biochemical composition such that the biochemical composition indicator represents the first biochemical composition, and
wherein the processor is further configured to provide, on the display, a second biochemical composition indicator representing a different, second biochemical composition that is present within the blood vessel.

13. The system of claim 1, wherein the intravascular device comprises an imaging catheter.
